(19) United States
(12) Patent Application Publication
(10) Pub. No.: US 2013/0046137 A1
(43) Pub. Date: Feb. 21, 2013

(54) SURGICAL INSTRUMENT AND METHOD WITH MULTIPLE IMAGE CAPTURE SENSORS

(71) Applicants: Tao Zhao, Sunnyvale, CA (US); Simon P. DiMaio, Sunnyvale, CA (US); David W. Bailey, Portola Valley, CA (US); Amy E. Kerdok, San Jose, CA (US); Gregory W. Dachs, II, San Francisco, CA (US); Stephen J. Blumenkranz, Redwood City, CA (US); Austin Reiter, New York, NY (US); Christopher J. Hasser, Los Altos, CA (US)

(72) Inventors: Tao Zhao, Sunnyvale, CA (US); Simon P. DiMaio, Sunnyvale, CA (US); David W. Bailey, Portola Valley, CA (US); Amy E. Kerdok, San Jose, CA (US); Gregory W. Dachs, II, San Francisco, CA (US); Stephen J. Blumenkranz, Redwood City, CA (US); Austin Reiter, New York, NY (US); Christopher J. Hasser, Los Altos, CA (US)

(73) Assignee: Intuitive Surgical Operations, Inc., Sunnyvale, CA (US)

(21) Appl. No.: 13/210,123

(22) Filed: Aug. 15, 2011

(51) Int. Cl. A61B 1/05; A61B 1/045; A61B 1/012

(52) U.S. Cl. 600/102; 600/109; 600/104

(57) ABSTRACT

A surgical instrument has a distal end portion with an outer surface with an outer radius. One or more image capture elements are movably mounted in the distal end portion. In a first state, the one or more image capture elements are un-deployed. In the first state, a surface having an aperture of at least one of the one or more image capture elements is enclosed within the outer surface of the surgical instrument so that the surface having the aperture does not extend beyond the outer surface. In a second state, the one or more image capture elements are deployed. In the second state the surface having the aperture of the at least one of the one or more image capture elements extends beyond the outer surface.

### BACKGROUND

1. Field of Invention

Aspects of this invention are related generally to surgical image capture sensors and are more particularly related to a minimally invasive surgical instrument with image capture sensors.

2. Related Art

The da Vinci® Surgical System, commercialized by Intuitive Surgical, Inc., Sunnyvale, Calif., is a minimally invasive teleoperated surgical system that offers patients many benefits, such as reduced trauma to the body, faster recovery and shorter hospital stay. One key component of the da Vinci® Surgical System is a capability to provide two-channel (i.e., left and right) video capture and display of visible images to provide stereoscopic viewing for the surgeon. Such electronic stereoscopic imaging systems may output high definition video images to the surgeon, and may allow features such as zoom to provide a “magnified” view that allows the surgeon to identify specific tissue types and characteristics, as well as to work with increased precision. The stereoscopic display gives the surgeon three-dimensional immersion and the ability to assess the depth of field.

Typically in a minimally invasive surgical system, an image capture system is coupled to a proximal end of a stereoscopic endoscope. The stereoscopic endoscope typically views a very small area. Areas that are outside the field of view of the endoscope cannot be monitored without repositioning the endoscope.

Another issue that has been recognized with a conventional endoscope or laparoscope is sterilization of the devices. One solution to this problem was to provide a pluggable opto-electronic module on a distal tip of surgical instrument so that the conventional endoscope was not needed. FIG. 1 is an example from U.S. Patent Application Publication No. US 2009/0318758 A1 (filed Mar. 27, 2009; disclosing Pluggable Vision Module and Portable Display for Endoscopy).

Cannula 100 (FIG. 1) is inserted into the body at an opening or incision 103. An illumination module 140, e.g., color light emitting diodes (LEDs), is mounted on a distal end surface 105 of cannula 100. An opto-electronic vision module 150 is also mounted on distal end surface 105. Distal end 102 of cannula 100 is flexible so that after insertion into the body, distal end 102 of cannula can be expanded radially by passing a surgical instrument through cannula 100. Electrical power to illumination module 140 and opto-electronic vision module 150 is provided by flexible electrical lines 104 that run along the cannula body and terminate at an electrical connector 106 at or near proximal opening 108 of cannula 100.

A flexible electrical cable 155 is connected between connector 106 and a portable control and display unit 170. Cable 155 transfers power and control signals to illumination module 140 and to opto-electronic vision module 150 from portable control and display unit 170, while providing imaging data from opto-electronic vision module 150 to the portable control and display 170. When cannula 100 is sterilized, illumination module 140 and opto-electronic vision module 150 are removed from cannula 100.

### SUMMARY

In one aspect, an apparatus includes a surgical instrument. The surgical instrument has a distal end portion. The distal end portion has an outer surface with an outer radius.

One or more image capture elements are movably mounted in the distal end portion. Each image capture element includes a surface having an aperture and an image capture sensor mounted in the image capture element. The image capture sensor is mounted so that the sensor captures light that passes through the aperture in the surface.

In a first state, the one or more image capture elements are un-deployed. In the first state, the surface having the aperture of at least one of the one or more image capture elements is enclosed within the outer surface so that the surface having the aperture does not extend beyond the outer surface. Thus, unlike the prior art devices, the image capture sensors are not exposed during the insertion of the surgical device.

In a second state, the one or more image capture elements are deployed. In the second state, the surface having the aperture of the at least one of the one or more image capture elements is positioned beyond the outer radius of the outer surface and so extends beyond the outer surface.

In one aspect, at least one image capture element also includes an illuminator. The illuminator is one or more light emitting diodes, in one example. The illuminator can be powered by an external power source, or a power source included within the surgical instrument.

The surgical instrument also includes a hinge element. The hinge element connects one image capture element in the one or more image capture elements to the distal end portion of the surgical instrument. The surgical instrument further includes an orientation marker fixed in position relative to the one or more image capture elements. The surgical instrument still further includes a proximal end portion with a clamp engagement structure positioned on the proximal end portion.

In one aspect, the surgical instrument is a cannula with an obturating tip. The obturating tip is made up of a plurality of distal end puncture tip elements. Each of the puncture tip elements is movably connected to the distal end portion of the cannula.

In another aspect, the surgical instrument is a cannula. A sheath extends through the cannula. The sheath includes the one or more image capture elements.

The apparatus also includes an elongate rod with an illuminator. Upon passing the elongate rod through the surgical instrument, the one or more image capture elements move from the first state to the second state. The apparatus further includes a controller coupled to each image capture element in the one or more image capture elements. The controller generates a panoramic image from images captured by the one or more image capture elements. The apparatus still further includes a portable display unit attached to the elongate rod proximal to the surgical instrument.

In another aspect, the apparatus includes an endoscope. Upon passing the endoscope through the surgical instrument, the one or more image capture elements move from the first state to the second state. This aspect also includes a controller coupled to each image capture element in the one or more image capture elements and to the endoscope. The controller generates a panoramic image from images captured by the one or more image capture elements. The controller sends an image captured from the endoscope and the panoramic image to a display device. In one aspect, the panoramic image includes a footprint of the image captured from the endoscope. In another aspect, the panoramic image and image captured from the endoscope are blended together by the controller and then sent to the display device.

In still another aspect, the apparatus includes a portable display unit attached to a proximal end portion of the surgical instrument.

In one aspect, a method includes positioning a surgical instrument. The surgical instrument includes one or more image capture elements in an un-deployed state. In the un-deployed state, an image capture sensor in at least one of the one or more image captures elements is proximal to a distal end surface of the surgical instrument and enclosed within an outer diameter of the instrument.

In this method, a device is inserted through the surgical instrument to deploy the one or more image capture elements. Also, a panoramic image is generated from images captured by the one or more deployed image capture elements.

The method includes placing a port in a patient using the panoramic image. The method also includes engaging the surgical instrument to a manipulator arm of a minimally invasive surgical system to orient the one or more image capture elements in a known orientation.

In one aspect, the inserting a device includes inserting an elongate rod including an illuminator through the surgical instrument so that a distal end of the elongate rod extends beyond a distal end of the surgical instrument.

In another aspect, the inserting a device includes inserting an endoscope through the surgical instrument so that a distal end of the endoscope extends beyond a distal end of the surgical instrument. The method then includes combining an image captured from the endoscope with the panoramic image with an orientation of the panoramic image rotated to the orientation of the image captured from the endo scope in a first combination image. The first combination image is displayed on a display for an operator of the endoscope. The method also includes a combining an image captured from the endoscope with the panoramic image with an orientation of the image captured from the endoscope rotated to the orientation of the panoramic image in a second combination image. The second combination image is displayed on a display different from the display used by an operator of the endoscope.

The method also includes blending an image captured from the endoscope with the panoramic image. In another aspect, the method indicates a footprint of an image captured from the endoscope in the panoramic image.

### BRIEF DESCRIPTION OF THE DRAWINGS

FIG. 1 is an illustration of a prior art cannula.

FIG. 2A is an illustration of surgical instrument that includes one or more image capture elements with the image capture elements automatically positioned in a first state.

FIG. 2B is an illustration of the surgical instrument of FIG. 2A with the image capture elements automatically positioned in a second state by passage of a device through a central channel in the surgical instrument.

FIG. 3 is a block diagram of a minimally invasive surgical system that includes the surgical instrument of FIGS. 2A and 2B.

FIGS. 4A to 4C are illustrations of various displays including a panoramic image generated from images captured using the surgical instrument of FIGS. 2A and 2B.

FIG. 5A is a cut-away illustration of a first cannula that includes one or more image capture elements with the image capture elements automatically positioned in a first state.

FIG. 5B is a cut-away illustration of the cannula of FIG. 5A with the image capture elements automatically positioned in a second state by passage of a device through a central channel in the cannula.

FIG. 6A is a cut-away illustration of a second cannula that includes one or more image capture elements with the image capture elements automatically positioned in a first state.

FIG. 6B is a cut-away illustration of the cannula of FIG. 6A with the image capture elements automatically positioned in a second state by passage of a device through a central channel in the cannula.

FIG. 7A is a cut-away illustration of a third cannula that includes one or more image capture elements with the image capture elements automatically positioned in a first state.

FIG. 7B is a cut-away illustration of the cannula of FIG. 7A with the image capture elements automatically positioned in a second state by passage of a device through a central channel in the cannula.

FIG. 8A is a cut-away illustration of a cannula used with a sheath that includes one or more image capture elements with the image capture elements automatically positioned in a first state.

FIG. 8B is a cut-away illustration of the cannula of FIG. 8A with the sheath that includes one or more image capture elements with the image capture elements automatically positioned in a first state positioned in the cannula.

FIG. 8C is a cut-away illustration of the cannula and sheath of FIG. 8B with the image capture elements automatically positioned in a second state by passage of a device through a central channel in the cannula.

FIG. 8D is an end view of the cannula, sheath, and device of FIG. 8C.

FIG. 9A is an illustration of an insertion lock inserted in a cannula with an obturating tip. The cannula includes one or more image capture elements with the image capture elements automatically positioned in a first state.

FIG. 9B is a top view of a distal tip of the insertion lock of FIG. 9A.

FIG. 9C is a side view of the distal tip of the insertion lock of FIG. 9A.

FIG. 9D is an illustration of the cannula with an obturating tip of FIG. 9A with the insertion lock removed.

FIG. 9E is an illustration of the cannula with an obturating tip of FIGS. 9A and 9D with the image capture elements automatically positioned in a second state by passage of a device through a central channel in the cannula.

FIG. 10 is an illustration of an alternative cannula, which is equivalent to the cannula of FIGS. 5A and 5B with the most distal portion of that cannula removed.

FIG. 11A is an illustration of a cannula that includes one or more yin-yang like image capture elements with the image capture elements automatically positioned in a first state.

FIG. 11B is an illustration of the cannula of FIG. 11A with the image capture elements automatically positioned in a second state by passage of a device through a central channel in the cannula

In the drawings, the first digit of a reference number indicates the figure in which the element with that reference number first appeared.

### DETAILED DESCRIPTION

Aspects of this invention facilitate acquiring images that can be used to construct a wide field of view image of a surgical sited. The wide field of view image, i.e., a panoramic image, can be used to insert a port into a patient without the need for a minimally invasive teleoperated camera or without the need for a separate laparoscope. In addition, as explained more completely below, the panoramic image can be combined with an image from an endoscope or laparoscope to provide information that cannot be obtained without moving the endoscope or laparoscope.

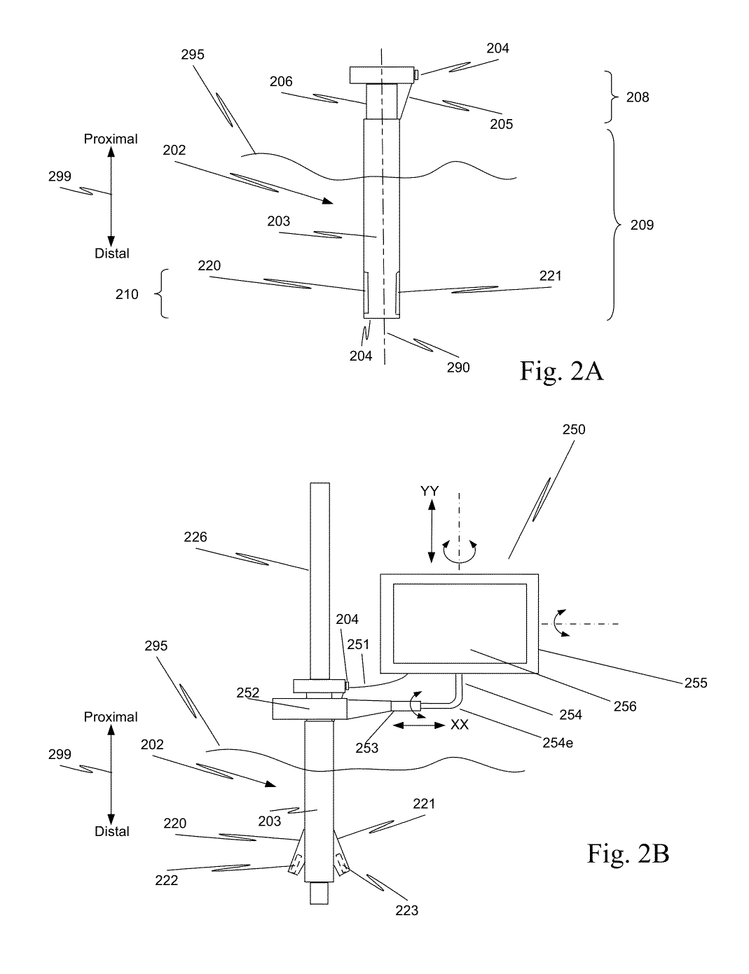

A surgical instrument 202 (FIGS. 2A, 2B), such as cannula or a guide tube, includes one or more image capture elements 220, 221 movably mounted on a distal end portion 210 of a body 209 of surgical instrument 202. As indicated by arrow 299, the distal direction is closer to the surgical site and the proximal direction is further away from the surgical site.

Typically, each image capture element 220, 221 includes an image capture sensor and a lens. The images captured by the image capture sensors are stitched together to generate the panoramic image.

Unlike the prior art system where the illumination and image capture sensors were mounted on the external surface of the distal end of the surgical instrument, image capture sensors 222, 223 (FIG. 2B) of image capture elements 220, 221, respectively, and any illumination devices are internal to surgical instrument 202 and are not exposed when surgical instrument 202 is inserted into a patient 295.

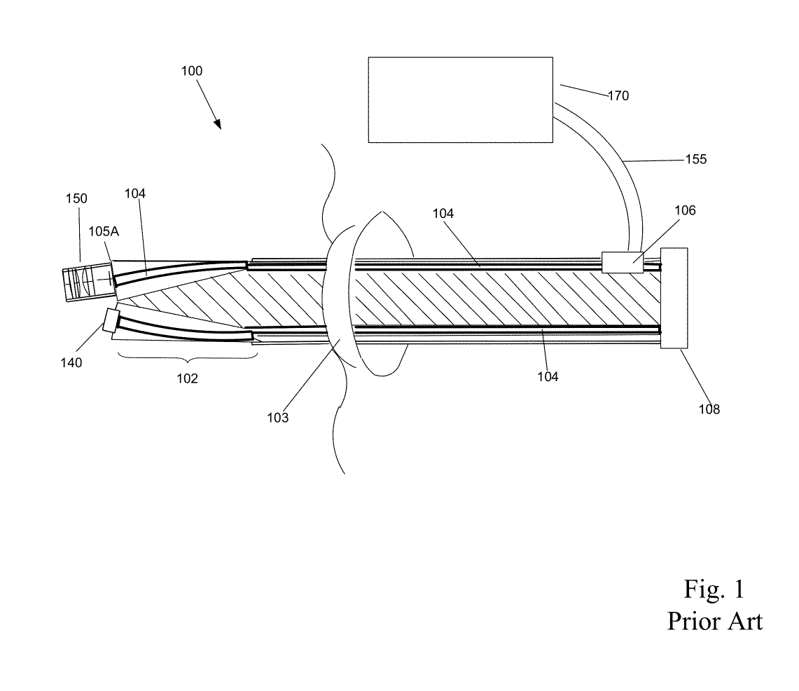

Unlike prior art structures (See FIG. 1), image capture elements 220, 221 are proximal to distal end surface 204 of surgical instrument 202. As illustrated in FIG. 2A initially, image capture elements 220, 221 are positioned in a first state, e.g., are un-deployed. In the first state, image capture sensors 222, 223 in image capture elements 220, 221 are contained within outer circumferential surface 203 of body 209. Thus, surgical instrument 202 has an outer diameter and a length similar to conventional devices. Surgical instrument 202, in the first state, does not have elements protruding from outer circumferential surface 203 or from distal end surface 204 that must be accommodated during insertion of surgical instrument 202 into patient 295.

To deploy image capture elements 220, 221, a device 226, e.g., a rod, a rod including an illuminator, an endoscope, or other surgical instrument, is passed through surgical instrument 202. As device 226 passes through surgical instrument 202, image capture elements 220, 221 are automatically displaced radially outward from a longitudinal axis 290 of surgical instrument 202 so that portions of image capture elements 220, 221 extend out from body 209 beyond outer circumferential surface 203.

In one aspect, each of image captures elements 220, 221 includes an image capture sensor 222, 223, a lens, and optionally at least one illumination device. Each of image capture sensors 222, 223 captures a different image. In one aspect, cables run from image capture sensors 222, 223 and run from any illumination devices within the wall of surgical instrument 202 to a power, control, and video interface 204. In another aspect, cables run from image capture sensors 222, 223 and run from any illumination devices through grooves formed in outer circumferential surface 203 to power, control, and video interface 204. Also, a combination of channels within the wall and surface groves could be used to route the cable between image capture sensors 222, 223 and any illumination devices to power, control, and video interface 204.

Power, control, and video interface 204 is illustrative only and is not intended to be limiting to this specific aspect. In view of this disclosure, a power source could be provided in surgical instrument 202. Also, the control and video signals could be transmitted to and from surgical instrument 202 using wireless communications, for example.

In the example of FIG. 2B, a lightweight portable display unit 250 is affixed to surgical instrument 202 by an attachment mechanism 252. Alternatively, lightweight portable display unit 250 can be affixed to device 226 proximal to surgical instrument 202 by attachment mechanism 252.

Portable display unit 250 includes a display device 255 that is moveably coupled to attachment mechanism 252 so that the orientation and location of display device 255 is easily changed. A power and video cable 251 of portable display unit 250 is connected to power, control, and video interface 204.

The images captured by image capture sensors 222, 223 in image capture elements 220, 221 are displayed on a display device 255 of portable display unit 250 as a panoramic view. Portable display unit 250 includes components and modules that convert the image data from image capture sensors 222, 223 into a wide field of view image, i.e., a panoramic image. Portable display unit 250 also includes components and modules to control image capture sensors 222, 223 as well as any illumination devices included in image capture elements 220, 221. The illumination provided either by an illuminator in device 226, illuminators in image captures elements 220, 221, or the combination of such illuminators is bright enough to provide trans illumination to ensure that no surface vessels are in the way of port placement.

The panoramic image on display device 255 can be used, for example, in placement of a port into patient 295. If a different view is needed, surgical instrument 202 can be rotated about its longitudinal axis 290 to provide a different panoramic image on display 256 of display device 255.

When placing a port under direct visual guidance using surgical instrument 202, there is no need to use a minimally invasive camera unit or a separate laparoscope. As noted above, the illumination obtained using surgical instrument 202 is bright enough to allow trans illumination of the patient's skin, which aids in ensuring that no surface blood vessels are in the way of the port placement. In addition, surgical instrument 202 provides a larger visual field than can typically be obtained by manipulating such conventional devices for the port placement under direct visual guidance. This provides better safety in port placement and minimizes the time required for port placement compared to prior art techniques.

The number of image capture sensors and illumination devices included in an image capture element is a function of at least the size of these devices. In another aspect, an image capture element includes only an image capture sensor. When the image capture elements include only image capture sensors, an illumination device may be mounted on or in the distal end of the surgical instrument. In still another aspect, an image capture element includes two or more image capture sensors. In some aspects, an image capture element may include only one or more illumination devices. Also, in some aspects, the image capture elements function as cannula retention devices. In addition, in some aspects, the displacement of the image capture units from the central axis of the surgical axis upon deployment is selected to facilitate any one of or any combination of stitching captures images together to form a panoramic view, as explained more completely below, to provide a desired resolution, and to minimize inadvertent tissue contact.

When surgical instrument 202 includes a plurality of image capture elements, images captured by a pair of the image capture elements can be used to generate a stereoscopic image. Irrespective of the number of image capture elements included in surgical instrument 202, the images captured by the image capture elements provide a wider field of view than was possible using a conventional endoscope or laparoscope, for example.

Returning to FIG. 2A, a proximal portion 208 of surgical instrument 202 includes an engagement structure 206 and an orientation marker 205. Body 209 extends distally from proximal portion 208.

Engagement structure 206 is configured so that surgical instrument 202 can be mounted in a minimally invasive surgical system in one aspect. In another aspect, engagement structure 206 is configured to facilitate using surgical instrument in a system of interest.

Orientation marker 205 is positioned in fixed relationship to image capture elements 220, 221. This permits viewing orientation marker 205, which is external to a patient, and determining the orientation of image capture elements 220, 221, which may be inside the patient and not visible. In one aspect, orientation marker 205 is a key that extends from the proximal end of body 209 to the proximal end of engagement structure 206. In the aspects of FIGS. 2A and 2B, orientation marker 205 is a wedge-shaped structure.

Display device 255 of a portable display unit 250 (FIG. 2B) is movable in multiple directions to facilitate positioning display 256 so that the displayed images are easily viewed. In this aspect, a second arm 254 moves longitudinally into and out of a first arm 253 that extends from attachment mechanism 252. Second arm 254 is also rotatable within first arm 253. Hence, first and second arms 253, 254 are a first telescopic mechanism that expands and contracts in a first direction XX, and that permits rotation of a second part of the mechanism relative to a first part of the mechanism.

Second arm 254 is part of a second telescopic mechanism that expands and contracts in a second direction YY that is different from first direction XX. In this example, second direction YY is perpendicular to first direction XX. Also, in this aspect, arm 254 includes a ninety-degree bend, e.g., an elbow, that separates the first telescopic mechanism from the second telescopic mechanism. In one aspect, elbow 254e is a flexible joint that provide additional ranges of motion. At the end of second arm 254 removed from elbow 252e and adjacent display device 255 is a two-dimensional joint that permits rotation of display device 255 around a longitudinal axis of arm 254 and rotation of display device 255 about an axis perpendicular to the longitudinal axis. In one aspect, when display device 255 is in the desired location, each of the movable elements is locked in position.

The particular method used to couple display device 255 to attachment mechanism 252 is not critical so long as display device 255 can be moved into a viewable position with minimal or no interference with the anatomy of the patient and with minimal or no interference with device 226 that is inserted through surgical instrument 202. The materials for elements in portable display unit 250 are selected to keep the weight of portable display unit 250 as low as possible to reduce the weight load on surgical instrument 202. Also, in one aspect, the positioning elements and display device 255 are counterbalanced by attachment mechanism 252 to reduce torques on surgical instrument 202.

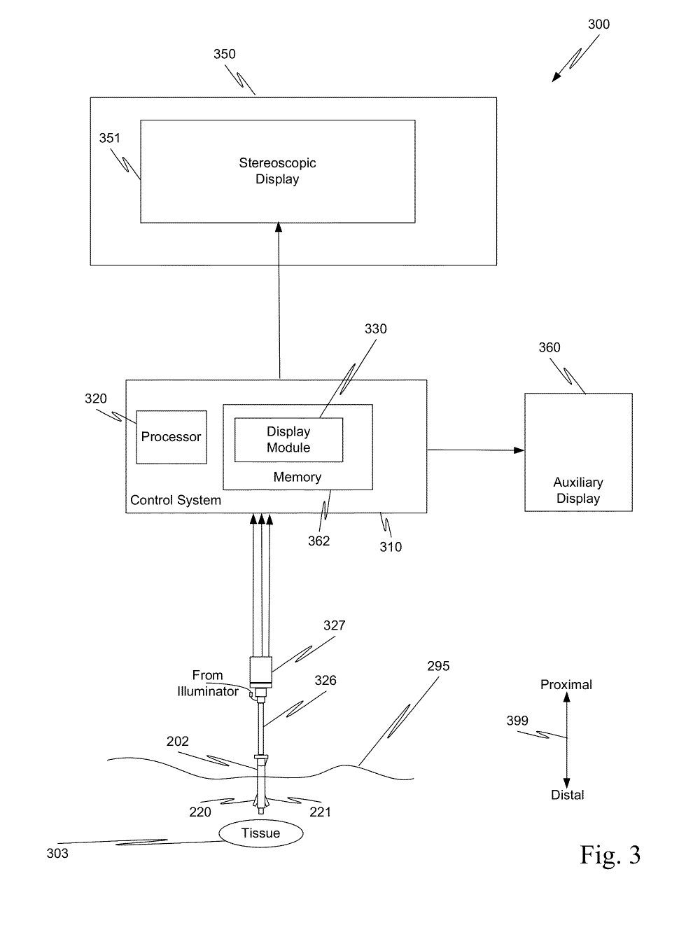

In another aspect illustrated in FIG. 3, an endoscope 326 is inserted through the central channel in surgical instrument 202 to deploy image capture elements 220, 221. As indicated by arrow 399, the distal direction is closer to the surgical site, e.g., tissue 303 and the proximal direction is further away from the surgical site.

Endoscope 326 is mounted in a minimally invasive surgical system 300, e.g., a da Vinci® minimally invasive teleoperated surgical system commercialized by Intuitive Surgical, Inc. of Sunnyvale, Calif. In this example, a surgeon at surgeon's console 350 remotely manipulates endoscope 326 that is mounted on a robotic manipulator arm (not shown). There are other parts, cables, etc. associated with the da Vinci® Surgical System, but these are not illustrated in FIG. 3 to avoid detracting from the disclosure. Further information regarding minimally invasive surgical systems may be found for example in U.S. patent application Ser. No. 11/762,165 (filed Jun. 23, 2007; disclosing Minimally Invasive Surgical System), U.S. Pat. No. 6,837,883 B2 (filed Oct. 5, 2001; disclosing Arm Cart for Telerobotic Surgical System), and U.S. Pat. No. 6,331,181 (filed Dec. 28, 2001; disclosing Surgical Robotic Tools, Data Architecture, and Use), all of which are incorporated herein by reference.

In one aspect, before inserting endoscope 326 into surgical instrument 202, instrument 226 (FIG. 2B) is withdrawn from surgical instrument 202. As instrument 226 is withdrawn, image capture elements 220, 221 automatically retract back into surgical instrument 202, e.g., automatically return to the first state. Thus, the operation of surgical instrument 202 in response to the insertion of endoscope 326 is the same irrespective of whether another device was inserted in and withdrawn from surgical instrument 202 prior to the insertion of endoscope 326.

Signals from image capture sensors 222, 223 in image capture elements 220, 221 are provided to a display module 330 that is executing on a processor 320 in a control system 310 of minimally invasive surgical system 300. Similarly, signals from image capture sensor(s) in image capture unit 327 attached to the proximal end of endoscope 326 are provided to executing display module 300.

Control system 310 controls the capture of images by endoscope 326 and by image capture sensors 222, 223. Control system 310 also controls any illumination devices in image capture elements 220, 221 as well as illumination provide to the illumination path in endoscope 326. Thus, control system 310 controls the illumination of tissue 303. Typically, when illumination is provided by endoscope 326, any illumination devices in image capture elements 220, 221 are turned off.

Display module 300 generates images for display on stereoscopic display unit 351 in surgeon's console 350, and optionally, for display on auxiliary display unit 360. The image or images displayed on stereoscopic display unit 351 and on auxiliary display unit 360 may be the same image or images, or may be a different image or images.

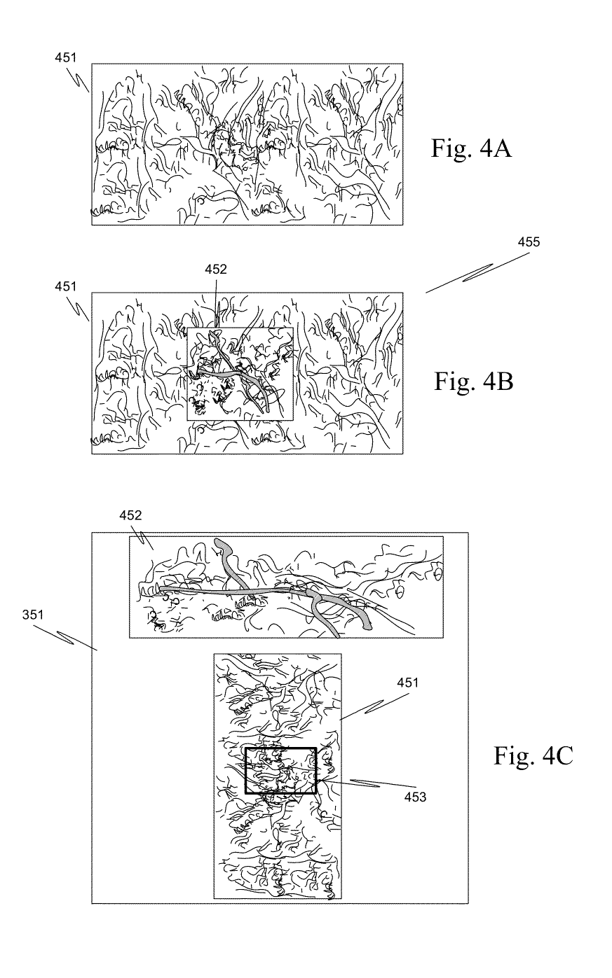

FIGS. 4A to 4C are examples of images that may be presented on stereoscopic display unit 351 and on auxiliary display unit 360. In FIG. 4A, images from image capture sensors 222, 223 in image capture elements 220, 221 are stitched together in a spatially consistent matter by display module 330 to form a single panoramic image 451 that is displayed. Panoramic image 451 can be generated by either the system in FIG. 2B, or the system in FIG. 3.

Panoramic image 451 provides a wide field of view. As indicated above, the wide field of view allows directing a newly inserted surgical instrument directly to the target anatomy. Previously, when the surgical instrument was inserted, a large part of the path to the target anatomy was un-monitored. There was a potential risk that the surgical instrument would damage a part of the anatomy while the surgical instrument was not visible to the main camera. To mitigate this risk, some surgeons moved the main camera to watch the path of the surgical instrument as it was inserted. Panoramic image 451 eliminates the need to move the main camera and so reduces the time required to insert the surgical instrument.

When surgical instrument 202 is the first cannula used, e.g., a camera cannula, panoramic image 451 enables placement of all the other ports without extra devices or extra personnel. Currently, most minimally invasive surgical systems use the main camera for placement of such ports. However, the main camera is heavy and big. This makes the main camera difficult to manipulate and usually one dedicated person needs to hold the camera. Sometimes, a smaller/lighter laparoscope is used. However, additional devices still are needed. Surgical instrument 202 does not have these limitations.

In FIG. 4B, combined image 455 includes an image 452 that was captured by the image capture unit attached to endoscope 326 combined with panoramic image 451. Combined image 455 is obtained in a picture in picture mode of operation. While no surgical instruments are illustrated in FIG. 4B, panoramic image 451 provides information on aspects of the surgical field that is out of the field of view of endoscope 326. Thus, the surgeon is provided information on surgical instruments that are not in the field of view of endoscope 326 as well as information on the anatomy of the patient that is not in the field of view of endoscope 326. Thus, panoramic image 451 effectively provides peripheral vision for the surgeon.

In one aspect, the image or images from endoscope 326 are pasted on panoramic image 451 in a spatially consistent matter. In another aspect, the image or images from endoscope 326 are blended with panoramic image 451 in a spatially consistent matter.

In one aspect, both endoscope 326 and image capture sensors 222, 223 in image capture elements 220, 221 are calibrated prior to using the instruments to obtain the focal lengths and other characteristics of endoscope 326 and image capture sensors 222, 223. One way display module 330 stitches the images together in a spatially consistent matter is to treat all the images as being part of a scene that is on a plane at a known distance from the end of endoscope 326 and then using known geometrical relationships to stitch the images using a plane perspective transformation.

For example, the plane distance is estimated by image matching. The plane distance for panoramic image 451 is estimated by matching the images from image capture sensors 222, 223. The plane distance for image 452 is estimated by matching the left and right images from stereoscopic endoscope 326.

In yet another aspect, the stitching parameters are at least partially estimated by detecting and matching visual features between images. For example, the stitching parameters are partially estimated by detecting and matching visual features between the images from image capture sensors 222, 223. Alternatively, the stitching parameters are partially estimated by detecting and matching visual features between the images from image capture sensors 222, 223 and the images from stereoscopic endoscope 326. The stitching of the images from image capture sensors 222, 223 and the images from stereoscopic endoscope 326 may also be done by approximating the actual transformation with an affine transformation, a similarity transformation, a rigid transformation, or a translation.

In one aspect, the surgeon can adjust the focus of combined image 455 if combined image 455 appears be out of focus. In an interface presented on display 351 in surgeon's console 350, a focus slider is presented to the surgeon. If initial combined image 455 does not appear properly focused, the surgeon can adjust the focus by changing the position of the focus slider. In response to the signal generated by the focus slider, display module 330 generates a new combined image that is presented on stereoscopic display 351.

In one aspect, to generate the new combined image in response to the signal from the focus slider, display module 330 uses information from a look-up table to adjust the focus. The lookup table includes data used to adjust the distance of the plane, e.g., the image depth, for the combined image based on the known geometrical relationships and characteristics of the image capture sensors and the cameras used to capture the received light from endoscope 326.

In another aspect, display module 330 generates a depth map using the images from endoscope 326 and uses the depth map to stitch the images together. In this aspect, if the images from endoscope 326 are not available, the field of views of the image capture sensors in image capture elements 220, 221 are designed to overlap sufficiently that the depth map can be generated using the overlapping portions of the images. The images from image capture elements 220, 221 can be stitched together using this depth map.

Display module 330 also blends the panoramic image and the image from endoscope 326 when generating combined image 455. For example, a portion of combined image 455 is made up of pixels in a central part of the image from endoscope 326 and no pixels from image capture elements 220, 221. Moving out from the central part of the image from endoscope 326, the pixel data in combined image 455 is an average of a pixel data from panoramic image 451 and pixel data from endoscopic image 452 with the weight given to panoramic image pixel data increasing as the process moves out from central part of the endoscopic image. For portions of combined image 455 that are outside of endoscopic image 452, only pixel data from panoramic image 451 is used.

For example, the endoscopic image is blended with the panoramic image using a transparency map for the endoscopic image, where the transparency is one hundred percent for the central part of the endoscopic image and then decreases to zero as the blending process moves away from the central part of endoscopic image to the periphery of endoscopic image. The transparency map is selected so that there is a smooth transition in contrast in moving from the central part of the endoscopic image to only data from the panoramic image. In one aspect, the transparency map and the determination of the central part of the endoscopic image is selected using empirical data from a number of viewers of the combined images generated using different transparency maps and different central parts.

In another aspect, to increase the dynamic range of panoramic image 451, the images from image capture elements 220, 221 are captured sequentially in time. A first shutter speed is used to capture the images from image capture elements 220, 221 and a second shutter speed is used to capture the images from endoscope 326. Typically, the objects in the field of view of endoscope 326 are illuminated more than the objects in the field of views of image capture sensors 222, 223 in image capture elements 220, 221. Thus, for this situation, the first shutter speed is slower than the second shutter speed. The set of sequential images are used to generate combined image 455 using the focal length and blending just described.

In yet another aspect, endoscopic image 452 and panoramic image 451 are both displayed on stereoscopic display 351, but images 451 and 452 are not combined. The side-by-side images are obtained in a side-by-side mode of operation.

For example in the side-by-side mode of operation, as illustrated in FIG. 4C, endoscopic image 452 is presented in a first portion of stereoscopic display 351, and panoramic image 451 presented in a second portion of stereoscopic display 351. The first portion is different from and removed from the second portion so that image 451 and image 452 are side-by-side, e.g., in one aspect do not overlap. To assist in indicating the location of endoscopic image 452 in panoramic image 451, a quadlateral 453 is positioned in panoramic image 451 to show the location of endoscopic image 452 in panoramic image 451, e.g., the foot print of endoscopic image 452 is pasted on panoramic image 451. The location of the footprint is determined using the same techniques as described above for the picture-in-picture mode of operation.

The examples in FIGS. 4A to 4C assumed that the same image or images was presented on both stereoscopic display 351 and auxiliary display 360 with the same layout and orientation of both panoramic image 451 and endoscopic image 452. However, in some aspects, the layout and orientation of the image or images displayed on stereoscopic display 351 and on auxiliary display 360 are not the same.

Typically, for the display of images on surgeon's console 350, the orientation of endoscopic image 452 is taken as defining the orientation for any images combined to form combined image 455. This orientation of endoscopic image 452 is known because control system 310 tracks the location and orientation of endoscope 326. Once surgical device 202 is mounted in minimally invasive surgical system 300, the positions and orientations of image capture sensors 222, 223 in image capture elements 220, 221 are fixed and known, which in turn defines the orientation of images captured by image capture sensors 222, 223.

Display module 330, in one aspect, generates panoramic image 451 and then rotates panoramic image 451 so that the orientation of panoramic image 451 is the same as the orientation of endoscopic image 452. The images or combined image presented on stereoscopic display 351 has the orientation of endoscopic image 452. The surgeon has independent control to switch on and off the display of panoramic image 451 on stereoscopic display 351.

The information needed by a surgeon's assistant when viewing an image on auxiliary display 360 may be different from the information needed by the surgeon viewing an image on stereoscopic display 351. For example, if the surgeon's assistant is inserting a new surgical instrument or port into the patient, the surgeon's assistant needs to know where left, right, up, and down are in the image on auxiliary display 360 relative to the patient. Thus, in one aspect, for the images or images displayed on auxiliary display 360, panoramic image 451 is fixed in position and the orientation of endoscopic image 452 is rotated by display module 330 to have the same orientation as the orientation of panoramic image 451. The surgeon's assistant can observe orientation marker 205 on surgical instrument 202 and can orient the image on display 360 to the patient based on the location of orientation marker 205 and the known position of the images devices relative to orientation marker 205. Thus, the surgeon's assistant can accurately ascertain left, right, up, and down in the image or images displayed on auxiliary display 360. The surgeon's assistant has independent control to switch on and off the display of panoramic image 451 on auxiliary display 360.

Thus, a surgical instrument 202, e.g., a cannula, a cannula having an obturating tip, a guide tube, includes one or more image capture sensors located on surgical instrument 202 that provide an auxiliary overall view of the internal anatomy of the patient. The image capture sensors are integrated on surgical instrument 202 as described above. This type of surgical instrument provides several different new capabilities.

For example, for port placement, there is no need to use an endoscope 326, or a separate laparoscope. This saves either personnel or the effort of using a separate laparoscope.

The panoramic image, i.e., the wide field of view, from surgical instrument 202 assists the surgeon's assistant in safety guiding an instrument to the target anatomy. The panoramic image also assists in safety passing in material (e.g., suture) to the target anatomy and safety taking out material (e.g., suture) from the body. The panoramic image also permits the surgeon's assistant to safely park materials (e.g., gauze) outside the field of view of endoscope 326 so that the materials are ready instantaneously when needed. In addition, the panoramic image provides better situation awareness outside the field of view of endoscope 326. The fixed position of the image capture sensors integrated in surgical device 202 permits a fixed roll angle of the image coordinate system for easy mental mapping of direction.

The peripheral vision provided by the panoramic image allows the surgeon to locate a lost instrument, such as a retractor, without repositioning endoscope 326. The panoramic image also allows the surgeon to anticipate a collision with instruments outside the field of view of endoscope 326. The panoramic image gives the surgeon better situational awareness outside the surgeon's main view from endoscope 326.

These new capabilities are provided by surgical instrument 202 with only minimal or no increase in the outer diameter of instrument 202 relative to a similar surgical instrument without these capabilities. The one or more image capture sensors are contained within this outer diameter prior to deployment and so are inside surgical instrument 202. The cylindrical wall of surgical instrument 202 only needs to provide passage for a video cable, a power cable, and any control cables for the one or more image capture sensors and any illumination devices included in image capture elements 220, 221.

The above examples used two image capture elements 220, 221 that each included an image capture sensor 222, 223. This is illustrative only and is not intended to be limiting. Surgical instrument 200 includes at least one image capture element with at least one image capture sensor and can include two or more image capture elements.

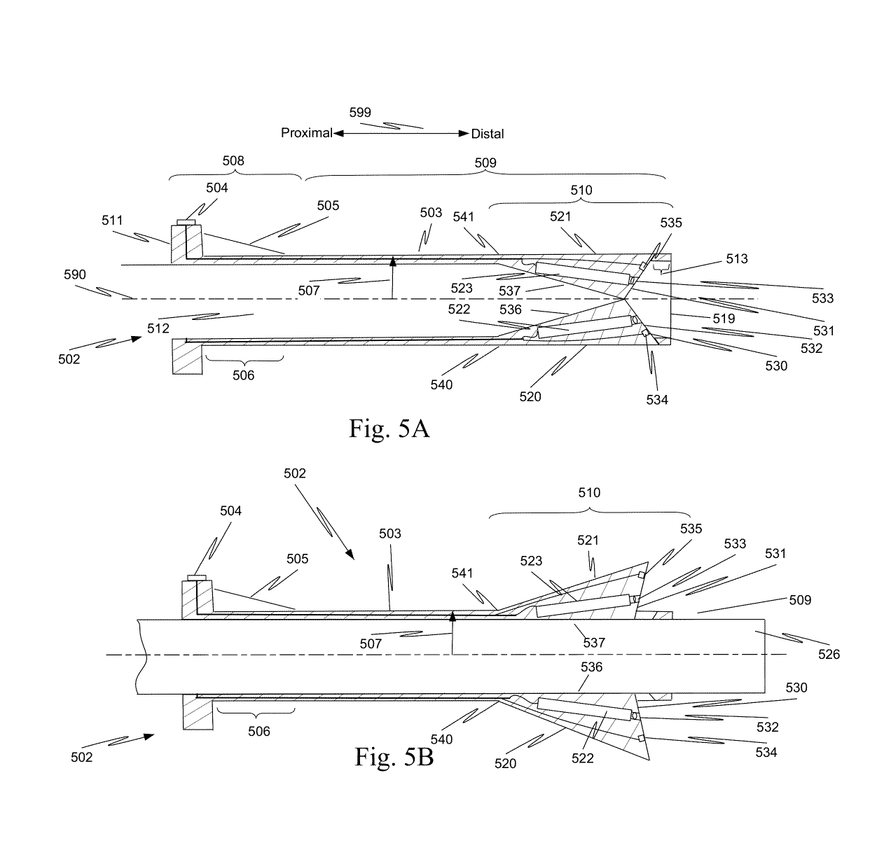

FIGS. 5A, 5B, 6A, 6B, 7A, 7B, 8A to 8D, 9A to 9E, 10, 11A and 11B illustrate alternative aspects of a surgical instrument with one or more image capture elements movably mounted on a distal end portion of a surgical instrument. In these aspects, each surgical instrument includes two image capture elements. Again, this is illustrative only and is not intended to be limiting to this specific configuration. For example, either less than two image capture elements, or more than two image capture elements can be used.

Also, in these figures, each image capture element includes a single image capture sensor and a lens, e.g., a camera, and an illumination device. Again, this is illustrative only and is not intended to be limiting to this specific configuration. For example, more than one image capture sensor could be used in an image capture element provided there is sufficient space. Also, an image capture element may include only an illumination device, only an image capture sensor, or any combination of the two devices.

When more than one image capture element with an image capture sensor is used, at least two of the image capture sensors in the image capture elements form a stereo pair (i.e., the two image capture sensors have sufficient overlap in their fields of view to create a stereoscopic image when viewed). The videos from these two image capture sensors are displayed in stereoscopic display 351 to enable three-dimensional perception by left/right eye disparity. The relative pose of the two image capture sensors is partially estimated by detecting and matching visual features of the two images. The wide field of view stereo image from the image capture sensors and the endoscopic stereo image are aligned so that their discontinuity in depth is minimized.

In FIGS. 5A and 5B, surgical instrument 202 is a cannula 502. Cannula 502 includes a proximal portion 508, and a body 509 extending distally from proximal portion 508. Body 509 includes a distal end portion 510. As indicated by arrow 599, the distal direction is towards a surgical site and the proximal direction is away from the surgical site.

Proximal portion 508 of cannula 502 includes power, control, and video interface 504, orientation marker 505, and engagement structure 506. Power, control, and video interface 504, orientation marker 505, and engagement structure 506 are equivalent to power, control, and video interface 204, orientation marker 205, and engagement structure 206, respectively, which were described above and so that description is incorporated herein by reference.

In this example, engagement structure 506 has the same outer diameter as body 509. Thus, cables from image capture sensors 522, 523 and illumination devices 534, 535 are routed within a channel in the cylindrical wall of distal end portion 510, body 509, and proximal portion 508 to collar 511 and then through collar 511 to power, control, and video interface 504. If engagement structure 506 has a smaller outer diameter than body 509, cables from image capture sensors 522, 523 and illumination devices 534, 535 are routed within a channel in the cylindrical wall of distal end portion 510, body 509, and orientation marker 505 to collar 511, in one aspect.

Orientation marker 505 can be other than the illustrated ramp structure. For example, the orientation marker could be a painted marker on collar 511. Also, more than one orientation marker 505 can be provided. For example, the orientation markers could be indents in the outer surface of engagement structure 506 that mate with a structure in the engagement device of the minimally invasive surgical system combined with markings on the outer circumferential surface of collar 511. Alternatively, power, control, and video interface 504 could be used as orientation marker 505. Thus, the number and implementation of orientation markers is not crucial so long as the orientation marker or markers permit correlation between the positions of the orientation markers and the orientation of image capture elements 520, 521.

In this example, body 509 has an outer circumferential surface 503 with an outer radius 507. Image capture elements 520, 521 are movably mounted on distal end portion 510. Image capture elements 520, 521 are connected to distal end portion 510 by hinge elements 540, 541, respectively. Hinge elements 540, 541 can be implemented, for example, using any one of a joint, a living hinge with a spring element, a flexure, and a pivot pin and a flexure element.

FIG. 5A illustrates hinge elements 540, 541 in the first state with image capture elements 520, 521 un-deployed. When there is not another device inserted through central channel 512 of cannula 502, hinge elements 540, 541 automatically maintain image capture elements 520, 521 in the first state. Hinge elements 540, 541 automatically return image capture elements 520, 521 to the first state when a device is withdrawn from central channel 512.

In this aspect, each image capture element 520, 521 includes an image capture sensor 522, 523, a lens, and an illumination device 534, 535. Image capture sensor 522, 523 and the lens are a small camera. Image capture sensor 522, 523 has a length longer than the diameter of cannula 502 and less than radius 507 of cannula 502. Radius 507 is the radius of outer circumferential surface 503.

The cameras used in image capture elements 520, 521 are similar to those available in cell phones and other small portable devices. The focal length of the camera is selected according to the desired field of view. The images captured by the camera typically have lower resolution than the images captured using an endoscope since the requirement on the resolution for peripheral vision is lower than the requirement on the resolution for fovea vision.

Image capture sensor 523 is positioned within image capture element 521 to capture light that passes through a first aperture 533 in edge surface 531 of image capture element 521. Typically, an aperture is filled with a window, a lens, a refracting component, or other optical component that passes light. Thus, an aperture is an opening in a surface that allows light to pass through the surface, and such openings include an opening that is filled with a window, a lens, a refracting component, or other optical component because such an opening still allows light to pass through.

Edge surface 531, image capture sensor 523, and illumination device 535 are proximal to distal end surface 519. In this aspect, distal end surface 519 is perpendicular to longitudinal axis 590 of cannula 502. In the first state, the edge of edge surface 531 closest to distal end surface 519 is positioned proximal to distal end surface 519 by a distance 513 along longitudinal axis 590. Edge surface 537 extends from hinge element 541 to an intersection with edge surface 531.

In the first state, edge surface 531, image capture sensor 523, and illumination device 535 do not extend beyond outer radius 507 of cannula 502 and so are enclosed within outer circumferential surface 503 of body 509. Thus, edge surface 531 having aperture 533 does not extend beyond outer circumferential surface 503 in the first state.

Similarly, image capture sensor 522 is positioned within image capture element 520 to capture light that passes through a first aperture 532 in edge surface 530 of image capture element 520. Edge surface 530, image capture sensor 522, and illumination device 534 are proximal to distal end surface 519. In the first state, the edge of edge surface 530 closest to distal end surface 519 is positioned proximal to distal end surface 519 by a distance 513 along longitudinal axis 590. Edge surface 536 extends from hinge element 540 to an intersection with edge surface 530.

In the first state, edge surface 530, image capture sensor 522, and illumination device 534 do not extend beyond outer radius 507 of cannula 502 and so are enclosed within outer circumferential surface 503 of body 509. Thus, edge surface 530 having aperture 532 does not extend beyond outer circumferential surface 503 in the first state. The enclosure of edge surface 531, image capture sensor 523, illumination device 535, edge surface 530, image capture sensor 522, and illumination device 534 within outer circumferential surface 503 means that the outer diameter and length of cannula 502 are similar to prior art cannulas that do not include image capture elements 520 and 521.

Illumination device 535 is positioned within image capture element 521 so that light from illumination device 535 passes through a second aperture in edge surface 531 of image capture element 521. Similarly, illumination device 534 is positioned within image capture element 520 so that light from illumination device 534 passes through with a second aperture in edge surface 530 of image capture element 520. In one aspect, each of illumination devices 534, 535 is one or more light emitting diodes.

When device 526 is passed through central channel 512 of cannula 502, (FIG. 5B) device 526 displaces image capture elements 520, 521 so that image capture sensors 522, 523 are moved to a position outside an inner radius of cannula 502. Aperture 533 in edge surface 531 is external to outer circumferential surface 503, i.e., any point on the border of aperture 533 is a distance greater than outer radius 507 from longitudinal axis 590. Also, aperture 532 in edge surface 530 is external to outer circumferential surface 503, i.e., any point on the border of aperture 532 is a distance greater than outer radius 507 from longitudinal axis 590. Edge surfaces 536, 537 are supported in a second state, i.e., a deployed state, by device 526. In the second state, edge surfaces 536, 537 are part of the inner wall of cannula 502.

Thus, image capture elements 520, 521 are deployed automatically by passing device 526 through cannula 502. Following deployment when the distal end of device 526 is moved proximal to hinge elements 540, 541, image capture elements 520, 521 automatically return to the first state without any action by a user of the minimally invasive surgical system, or by the control system of the minimally invasive surgical system in which cannula 502 is mounted.

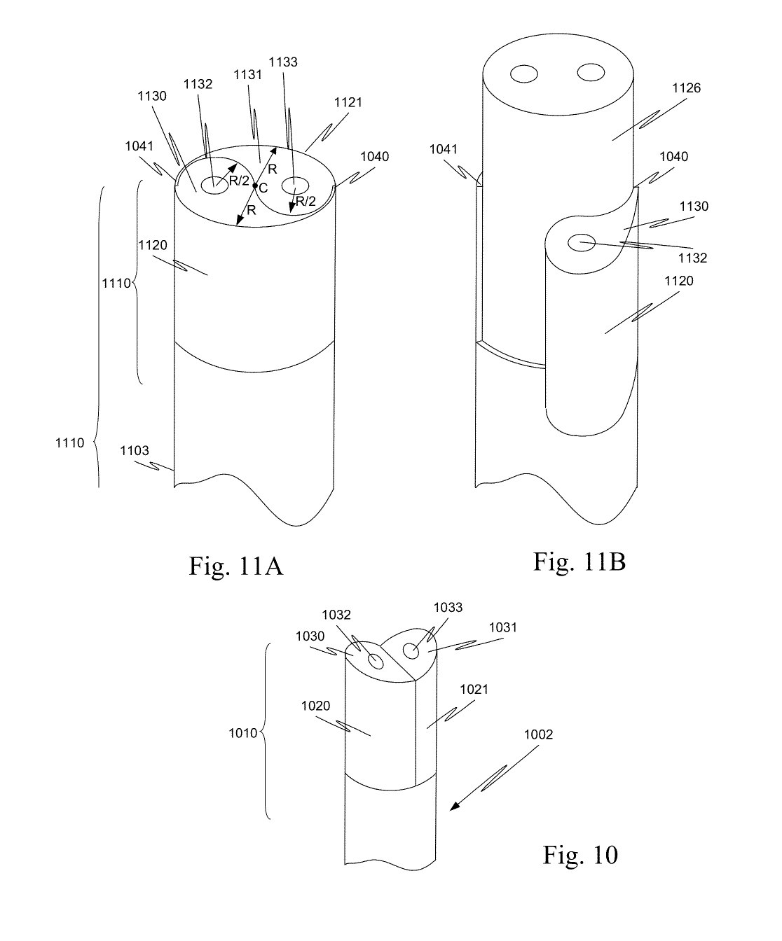

In another aspect, the part of distal end portion 510 having distance 513 along the outer surface of central channel 512 is eliminated. The resulting distal end portion 1010 of a cannula 1002 is illustrated in FIG. 10.

However, image capture elements 1020, 1021 in cannula 1002, in this aspect, do not include the illumination devices or the apertures for the illumination devices of cannula 502. As illustrated, in this aspect, image capture elements 1020, 102 include only apertures 1033, 1032 in edge surface 1031 and edge surface 1030, respectively. Each image capture element 1020, 1021 includes a lens and an image capture sensor. Each of the other features and the operation of cannula 1002 is equivalent to that just described for the features and operation with respect to cannula 502 and FIGS. 5A and 5B. Thus, to avoid repetition that description is not repeated for cannula 1002.

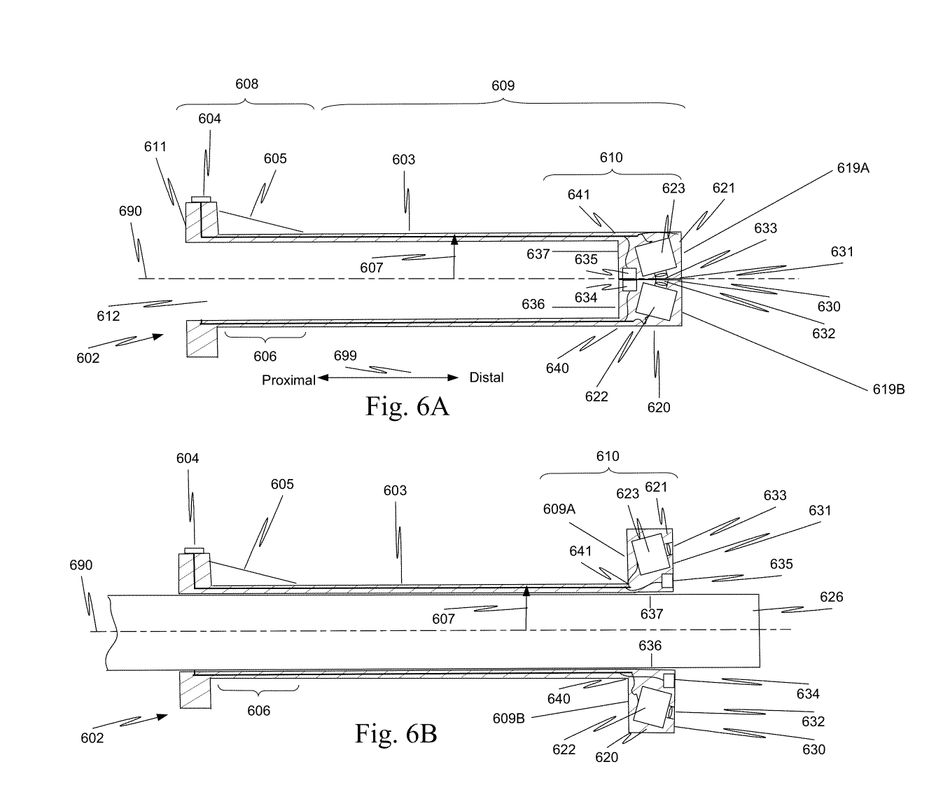

In FIGS. 6A and 6B, surgical instrument 202 is a second cannula 602. Cannula 602 includes a proximal portion 608, and a body 609 extending distally from proximal portion 608. Body 609 includes a distal end portion 610. As indicated by arrow 699, the distal direction is towards a surgical site and the proximal direction is away from the surgical site.

Proximal portion 608 of cannula 602 includes power, control, and video interface 604, orientation marker 605, and engagement structure 606. Power, control, and video interface 604, orientation marker 605, and engagement structure 606 are equivalent to power, control, and video interface 204, orientation marker 205, and engagement structure 206, respectively, which were described above and so that description is incorporated herein by reference.

In this example, engagement structure 606 has the same outer diameter as body 609. Thus, cables from image capture sensors 622, 623, and illumination devices 634, 635 are routed within a channel in the cylindrical wall of distal end portion 610, body 609, and proximal portion 608 to collar 611 and then through collar 611 to power, control, and video interface 604. If engagement structure 606 has a smaller outer diameter than body 609, cables from image capture sensors 622, 623, and illumination devices 634, 635 are routed within a channel in the cylindrical wall of distal end portion 610, body 609, and orientation marker 605 to collar 611, in one aspect.

Orientation marker 605 can be other than the illustrated ramp structure. For example, the orientation marker could be a painted marker on collar 611. Also, more than one orientation marker 605 can be provided. For example, the orientation markers could be indents in the outer surface of engagement structure 606 that mate with a structure in the engagement device of the minimally invasive surgical combined with markings on the outer circumferential surface of collar 611. Alternatively, power, control, and video interface 604 could be used as orientation marker 605. Thus, the number and implementation of orientation markers is not crucial so long as the orientation marker or markers permit correlation between the positions of the orientation markers and the orientation of image capture elements 620, 621.

In this example, body 609 has an outer circumferential surface 603 with an outer radius 607. Image capture elements 620, 621 are movably mounted on distal end portion 610. Image capture elements 620, 621 are connected to distal end portion 610 by hinge elements 640, 641, respectively. Hinge elements 640, 641 can be implemented, for example, using any one of a joint, a living hinge with a spring element, a flexure, and a pivot pin and a flexure element.

FIG. 6A illustrates hinge elements 640, 641 in the first state with image capture elements 620, 621 un-deployed. When a device is not inserted through central channel 612 of cannula 602, hinge elements 640, 641 automatically maintain image capture elements 620, 621 in the first state. Hinge elements 640, 641 automatically return image capture elements 620, 621 to the first state when a device is withdrawn from central channel 612.

In this aspect, each image capture element 620, 621 includes an image capture sensor 622, 623, lens, and an illumination device 634, 635. Image capture sensor 622, 623 and the lens is a small camera. Image capture sensor 622, 623 has a length less than outer radius 607 of cannula 602 and a width less than the outer diameter of cannula 602. Radius 607 is the radius of outer circumferential surface 603. The cameras used in image capture elements 620, 621 are similar to those described above.

Image capture sensor 623 is positioned within image capture element 621 to capture light that passes through a first aperture 633 in edge surface 631 of image capture element 621. Aperture 633, image capture sensor 623, and illumination device 635 are proximal to distal end surface 619A. In this aspect and in the first state, distal end surface 619A is perpendicular to longitudinal axis 690 of cannula 602. Distal end surface 619A could be shaped to form a desired angle with longitudinal axis 690, where the desired angle is different from ninety degrees.

Distal end surface 619A is an edge surface of image capture element 621 in this aspect. Edge surface 631 of image capture element 621 is along longitudinal axis 690 in the first state and so is substantially parallel to longitudinal axis 690. In this aspect and in the first state, edge surface 637 of image capture element 621 is perpendicular to longitudinal axis 690 and is substantially parallel to distal end surface 619A. Edge surface 637 extends from hinge element 641 to an intersection with edge surface 631. Edge surface 637 could also be oriented at an angle to longitudinal axis 690.

In the first state, edge surface 631, image capture sensor 623, and illumination device 635 do not extend beyond outer radius 607 of cannula 602 and so are enclosed within outer circumferential surface 603 of body 609. Thus, edge surface 631 having aperture 633 does not extend beyond outer circumferential surface 603 in the first state.

Similarly, image capture sensor 622 is positioned within image capture element 620 to capture light that passes through a first aperture 632 in edge surface 630 of image capture element 620. Aperture 632, image capture sensor 622, and illumination device 634 are proximal to distal end surface 619B. In this aspect and in the first state, distal end surface 619B is perpendicular to longitudinal axis 690 of cannula 602. Distal end surface 619B also could be shaped to form the desired angle with longitudinal axis 690, where the desired angle is different from ninety degrees.

Distal end surface 619B is an edge surface of image capture element 620 in this aspect. Edge surface 630 of image capture element 620 is along longitudinal axis 690 in the first state and so is substantially parallel to longitudinal axis 690. In this aspect and in the first state, edge surface 636 of image capture element 620 is perpendicular to longitudinal axis 690 and is substantially parallel to distal end surface 619B. Edge surface 636 extends from hinge element 640 to an intersection with edge surface 630. Edge surface 636 could also be oriented at an angle to longitudinal axis 690.

In the first state, edge surface 630, image capture sensor 622, and illumination device 634 do not extend beyond outer radius 607 of cannula 602 and so are enclosed within outer circumferential surface 603 of body 609. Thus, edge surface 630 having aperture 632 does not extend beyond outer circumferential surface 603 in the first state. The enclosure of edge surface 631, image capture sensor 623, illumination device 635, edge surface 630, image capture sensor 622 and illumination device 634 within outer circumferential surface 603 means that the outer diameter and length of cannula 602 is similar to prior art cannulas that do not include image capture elements 620 and 621.

Illumination device 635 is positioned within image capture element 621 so that light from illumination device 635 passes through a second aperture in edge surface 631 of image capture element 621. Similarly, illumination device 634 is positioned within image capture element 620 so that light from illumination device 634 passes through a second aperture in edge surface 630 of image capture element 620. In one aspect, each of illumination devices 634, 635 is one or more light emitting diodes.

When device 626 is passed through central channel 612 of cannula 602, (FIG. 6B) device 626 displaces image capture elements 620, 621 so that image capture sensors 622, 623 are moved to a position outside an inner radius of cannula 602. Aperture 633 in edge surface 631 is external to outer circumferential surface 603, i.e., any point on the border of aperture 633 is a distance greater than outer radius 607 from longitudinal axis 690. Also, aperture 632 in edge surface 630 is external to outer circumferential surface 603, i.e., any point on the border of aperture 632 is a distance greater than outer radius 607 from longitudinal axis 690. Edge surfaces 636, 637 are supported in a second state, i.e., a deployed state, by device 626. In the second state, edge surfaces 636, 637 are part of the inner wall of cannula 602.

Thus, image capture elements 620, 621 are deployed automatically by passing device 626 through cannula 602. Following deployment when the distal end of device 626 is moved proximal to hinge elements 640, 641, image capture elements 620, 621 automatically return to the first state without any action by a user of, or by the control system of the minimally invasive surgical system in which cannula 602 is mounted.

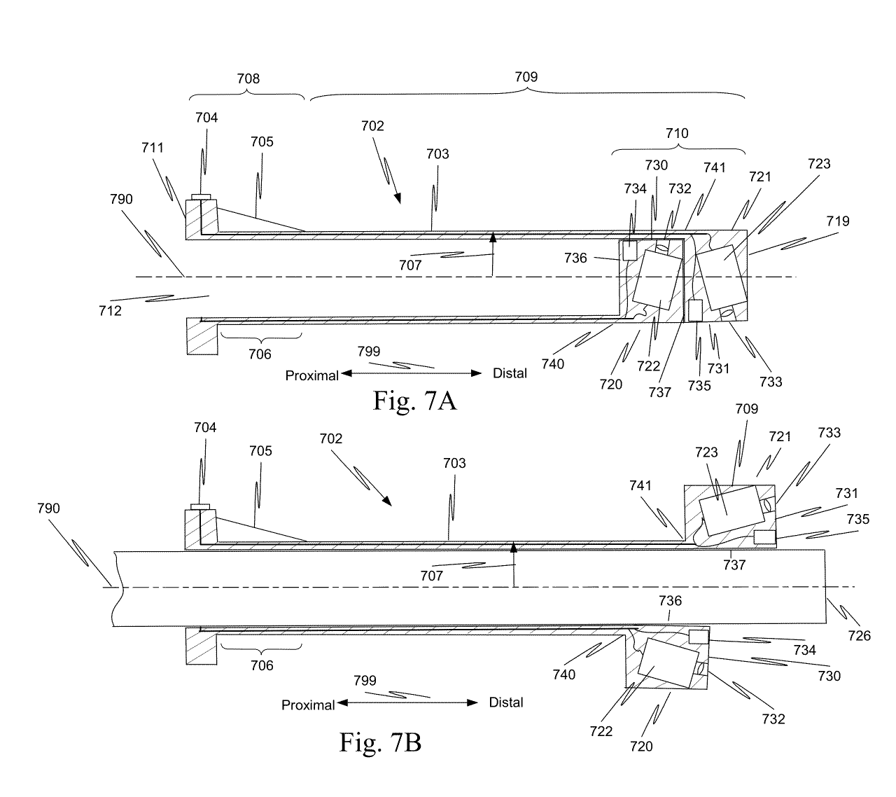

In FIGS. 7A and 7B, surgical instrument 202 is yet another cannula 702. Cannula 702 includes a proximal portion 708, and a body 709 extending distally from proximal portion 708. Body 709 includes a distal end portion 710. As indicated by arrow 799, the distal direction is towards a surgical site and the proximal direction is away from the surgical site.

Proximal portion 708 of cannula 702 includes power, control, and video interface 704, orientation marker 705, and engagement structure 706. Power, control, and video interface 704, orientation marker 705, and engagement structure 706 are equivalent to power, control, and video interface 204, orientation marker 205, and engagement structure 206, respectively, which were described above and so that description is incorporated herein by reference.

In this example, engagement structure 706 has the same outer diameter as body 709. Thus, cables from image capture sensors 722, 723, and illumination devices 734, 735 are routed within a channel in the cylindrical wall of distal end portion 710, body 709, and proximal portion 708 to collar 711 and then through collar 711 to power, control, and video interface 704. If engagement structure 706 has a smaller outer diameter than body 709, cables from image capture sensors 722, 723, and illumination devices 734, 735 are routed within a channel in the cylindrical wall of distal end portion 710, body 709, and orientation marker 705 to collar 711, in one aspect.

Orientation marker 705 can be other than the illustrated ramp structure. For example, the orientation marker could be a painted marker on collar 711. Also, more than one orientation marker 705 can be provided. For example, the orientation markers could be indents in the outer surface of engagement structure 706 that mate with a structure in the engagement device of the minimally invasive surgical combined with markings on the outer circumferential surface of collar 711. Alternatively, power, control, and video interface 704 could be used as orientation marker 705. Thus, the number and implementation of orientation markers is not crucial so long as the orientation marker or markers permit correlation between the positions of the orientation markers and the orientation of image capture elements 720, 721.

In this example, body 709 has an outer circumferential surface 703 with an outer radius 707. Image capture elements 720, 721 are movably mounted on distal end portion 710. Image capture elements 720, 721 are connected to distal end portion 710 by hinge elements 740, 741, respectively. Hinge elements 740, 741 can be implemented, for example, using any one of a joint, a living hinge with a spring element, a flexure, and a pivot pin and a flexure element.

FIG. 7A illustrates hinge elements 740, 741 in the first state with image capture elements 720, 721 un-deployed. When a device is not inserted through central channel 712 of cannula 702, hinge elements 740, 741 automatically maintain image capture elements 720, 721 in the first state. Hinge elements 740, 741 automatically return image capture elements 720, 721 to the first state when a device is withdrawn from central channel 712.

In this aspect, each image capture element 720, 721 includes an image capture sensor 722, 723, a lens, and an illumination device 734, 735. Image capture sensor 722, 723 and the lens is a small camera. Image capture sensor 722, 723 has a length and width larger than outer radius 707 of cannula 702, but smaller than the outer diameter of cannula 702. Radius 707 is the radius of outer circumferential surface 703. The cameras used in image capture elements 720, 721 are similar to those described above.

Image capture sensor 723 is positioned within image capture element 721 to capture light that passes through a first aperture 733 in edge surface 731 of image capture element 721. Aperture 733, image capture sensor 723, and illumination device 735 are proximal to distal end surface 719. In this aspect and in the first state, distal end surface 719 is perpendicular to longitudinal axis 790 of cannula 702. Distal end surface 719 could be other than a flat surface.

Distal end surface 719 is an edge surface of image capture element 721 in this aspect. Edge surface 731 of image capture element 721 is substantially parallel to longitudinal axis 790 in the first state and is included in the outer circumferential surface of cannula 702 in the first state. In this aspect and in the first state, edge surface 737 of image capture element 721 is perpendicular to and intersects longitudinal axis 790. Edge surface 737 also is substantially parallel to distal end surface 719. Edge surface 737 extends from hinge element 741 to an intersection with edge surface 731.

In the first state, image capture sensor 723, and illumination device 735 do not extend beyond outer radius 707 of cannula 702 and so are enclosed within outer circumferential surface 703 of body 709. Thus, edge surface 731 having aperture 733 does not extend beyond outer circumferential surface 703 and in the first state forms a part of surface 703.

Similarly, image capture sensor 722 is positioned within image capture element 720 to capture light that passes through a first aperture 732 in edge surface 730 of image capture element 720. Aperture 732, image capture sensor 722, and illumination device 734 are proximal to distal end surface 719. Edge surface 730 of image capture element 720 is substantially parallel to longitudinal axis 790 and adjacent an inner wall of cannula 702 in the first state. Also, in the first state, edge surface 736 of image capture element 720 is perpendicular to and intersects longitudinal axis 790. Edge surface 736 in this aspect is substantially parallel to distal end surface 719. Edge surface 736 extends from hinge element 740 to an intersection with edge surface 730.

In the first state, edge surface 730, image capture sensor 722, and illumination device 734 do not extend beyond outer radius 707 of cannula 702 and so are enclosed within outer circumferential surface 703 of body 709. Thus, edge surface 730 having aperture 732 does not extend beyond outer circumferential surface 703 in the first state. The enclosure of edge surface 730, image capture sensor 722, illumination device 734, image capture sensor 723 and illumination device 735 within outer circumferential surface 703 means that the outer diameter and length of cannula 702 is similar to prior art cannulas that do not include image capture elements 720 and 721.

Illumination device 735 is positioned within image capture element 721 so that light from illumination device 735 passes through a second aperture in edge surface 731 of image capture element 721. Similarly, illumination device 734 is positioned within image capture element 720 so that light from illumination device 734 passes through a second aperture in edge surface 730 of image capture element 720. In one aspect, each of illumination devices 734, 735 is one or more light emitting diodes.

When device 726 is passed through central channel 712 of cannula 702, (FIG. 7B) device 726 displaces image capture elements 720, 721 so that image capture sensors 722, 723 are moved to a position outside an inner radius of cannula 702. Aperture 733 in edge surface 731 is external to outer circumferential surface 703, i.e., any point on the border of aperture 733 is a distance greater than outer radius 707 from longitudinal axis 790. Also, aperture 732 in edge surface 730 is external to outer circumferential surface 703, i.e., any point on the border of aperture 732 is a distance greater than outer radius 707 from longitudinal axis 790. Edge surfaces 730 and 731 form part of the distal end surface of cannula 702 in the second state. Edge surfaces 736, 737 are supported in a second state, i.e., a deployed state, by device 726. In the second state, edge surfaces 736, 737 are part of the inner wall of cannula 702.

Thus, image capture elements 720, 721 are deployed automatically by passing device 726 through cannula 702. Following deployment when the distal end of device 726 is moved proximal to hinge elements 740, 741, image capture elements 720, 721 automatically return to the first state without any action by a user of, or by the control system of the minimally invasive surgical system in which cannula 702 is mounted.

In both the un-deployed and deployed states, image capture elements 720, 721 are different distances from proximal portion 708. For example, a distance from a point on proximal portion 708 to any point on edge surface 730 is smaller than a distance from the point on proximal portion 708 to any point on edge surface 731. This relationship is true for any part of image capture element 720 and the corresponding part of image capture element 721.

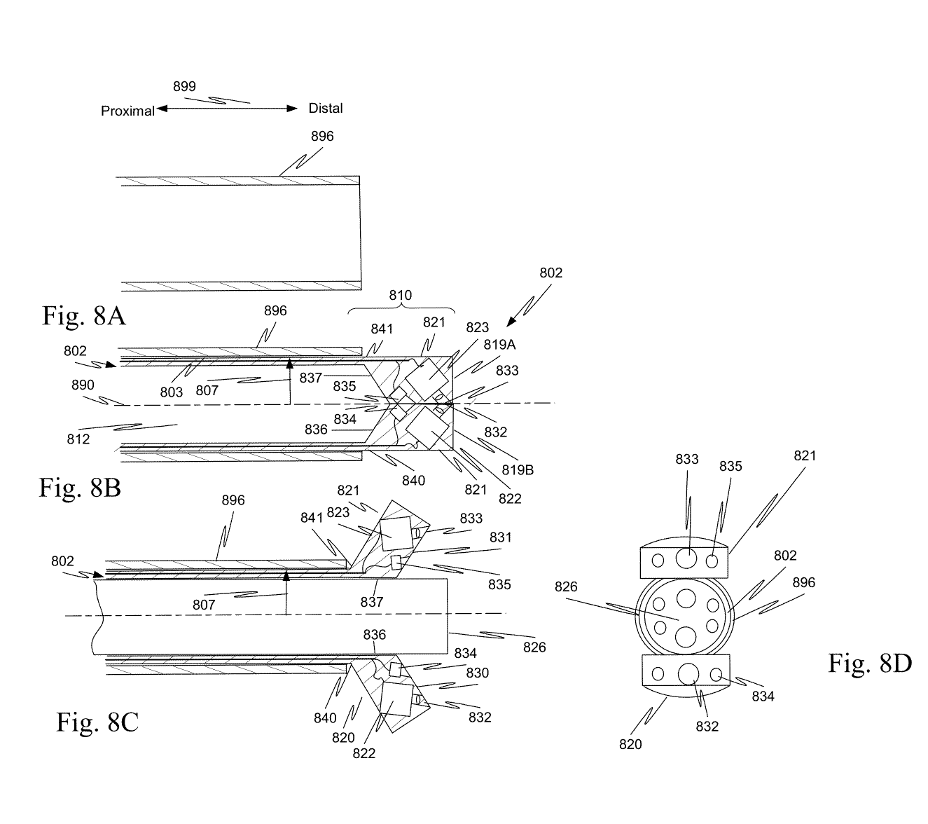

In FIGS. 8A to 8D, surgical instrument 202 is a combination of a cannula 896 and a sheath 802. Cannula 896 (FIG. 8A) is a conventional cannula. A sheath 802 (FIG. 8B) is inserted through cannula 896 to that a distal end portion 810 of a body of sheath 802 extends from the distal end of cannula 896. Sheath 802 also has a proximal portion similar to the proximal portions described above with an orientation marker and a power, control, and video interface. As indicated by arrow 899, the distal direction is towards a surgical site and the proximal direction is away from the surgical site.

In this example, the body of sheath 802 has an outer circumferential surface 803 with an outer radius 807. Image capture elements 820, 821 are movably mounted on distal end portion 810 of sheath 802. Image capture elements 820, 821 are connected to distal end portion 810 by hinge elements 840, 841, respectively. Hinge elements 840, 841 can be implemented, for example, using any one of a joint, a living hinge with a spring element, a flexure, and a pivot pin and a flexure element.

FIG. 8B illustrates hinge elements 840, 841 in the first state with image capture elements 820, 821 un-deployed. When a device is not inserted through central channel 812 of sheath 802, hinge elements 840, 841 automatically maintain image capture elements 820, 821 in the first state. Hinge elements 840, 841 automatically return image capture elements 820, 821 to the first state when a device is withdrawn from central channel 812.

In this aspect, each image capture element 820, 821 includes an image capture sensor 822, 823 and an illumination device 834, 835. Image capture sensor 822, 823 and the lens is a small camera such as those described above.

Image capture sensor 823 is positioned within image capture element 821 to capture light that passes through a first aperture 833 in edge surface 831 of image capture element 821. Aperture 833, image capture sensor 823, and illumination device 835 are proximal to distal end surface 819A. In this aspect and in the first state, distal end surface 819A is perpendicular to longitudinal axis 890 of sheath 802. Distal end surface 819A could be shaped to form a desired angle with longitudinal axis 890, where the desired angle is different from ninety degrees.

Distal end surface 819A is an edge surface of image capture element 821 in this aspect. Edge surface 831 of image capture element 821 is along longitudinal axis 890 in the first state and so is substantially parallel to longitudinal axis 890. In this aspect and in the first state, edge surface 837 of image capture element 821 intersects longitudinal axis 890 at an angle different from ninety degrees. Edge surface 837 extends from hinge element 841 to an intersection with edge surface 831. The angle of edge surface 837 can be changed to change the field of view of image capture sensor 823 in the second state that is described below.

In the first state, edge surface 831, image capture sensor 823, and illumination device 835 do not extend beyond outer radius 807 of sheath 802 and so are enclosed within outer circumferential surface 803 of sheath 802. Thus, edge surface 831 having aperture 833 does not extend beyond outer circumferential surface 803 in the first state.

Similarly, image capture sensor 822 is positioned within image capture element 820 to capture light that passes through a first aperture 832 in edge surface 830 of image capture element 820. Image capture sensor 822 and illumination device 834 are proximal to distal end surface 819B. In this aspect and in the first state, distal end surface 819B is perpendicular to longitudinal axis 890 of sheath 802.

Distal end surface 819B is an edge surface of image capture element 820 in this aspect. Edge surface 830 of image capture element 820 is along longitudinal axis 890 in the first state and so is substantially parallel to longitudinal axis 890. In this aspect and in the first state, edge surface 836 of image capture element 820 intersects longitudinal axis 890 at an angle different from ninety degrees. Edge surface 836 extends from hinge element 840 to an intersection with edge surface 830. The angle of edge surface 836 can be changed to change the field of view of image capture sensor 822 in the second state.

In the first state, edge surface 830, image capture sensor 822, and illumination device 834 do not extend beyond outer radius 807 of sheath 802 and so are enclosed within outer circumferential surface 803 of sheath 802. Thus, edge surface 830 having aperture 832 does not extend beyond outer circumferential surface 803 in the first state. Thus, edge surface 830 having aperture 832 does not extend beyond outer circumferential surface 803 in the first state. The enclosure of edge surface 831, image capture sensor 823, illumination device 835, edge surface 830, image capture sensor 822, and illumination device 834 within outer circumferential surface 803 means that the outer diameter of sheath 802 can be passed through cannula 896.

Illumination device 835 is positioned within image capture element 821 so that light from illumination device 835 passes through a second aperture in edge surface 831 of image capture element 821. Similarly, illumination device 834 is positioned within image capture element 820 so that light from illumination device 834 passes through a second aperture in edge surface 830 of image capture element 820. In one aspect, each of illumination devices 834, 835 is one or more light emitting diodes.

When device 826 is passed through central channel 812 of sheath 802 (FIG. 8C), device 826 displaces image capture elements 820, 821 so that image capture sensors 822, 823 are moved to a position outside an inner radius of sheath 802. Aperture 833 in edge surface 831 is external to outer circumferential surface 803, i.e., any point on the border of aperture 833 is a distance greater than outer radius 807 from longitudinal axis 890. Also, aperture 832 in edge surface 830 is external to outer circumferential surface 803, i.e., any point on the border of aperture 832 is a distance greater than outer radius 807 from longitudinal axis 890. Edge surfaces 836, 837 are supported in a second state, i.e., a deployed state, by device 826. In the second state, edge surfaces 836, 837 are part of the inner wall of sheath 802. FIG. 8D is an end view of cannula 896 and sheath 802 with image capture elements 820, 821 in the second state.

Thus, image capture elements 820, 821 are deployed automatically by passing device 826 through sheath 802. Following deployment when the distal end of device 826 is moved proximal to hinge elements 840, 841, image capture elements 820, 821 automatically return to the first state without any action by a user of, or by the control system of the minimally invasive surgical system in which sheath 802 is mounted.

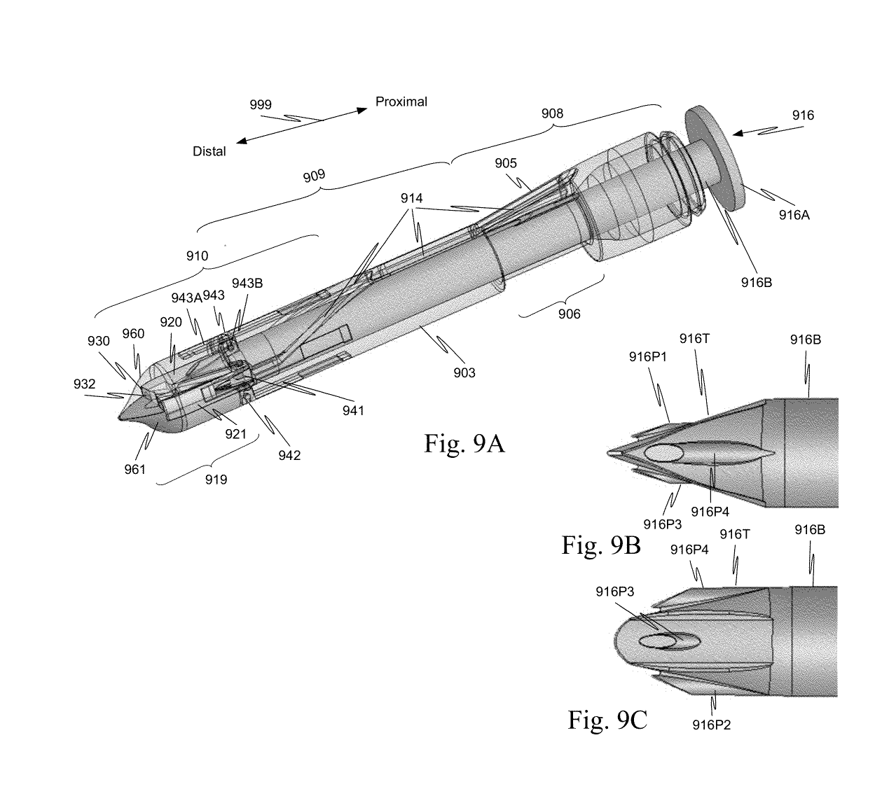

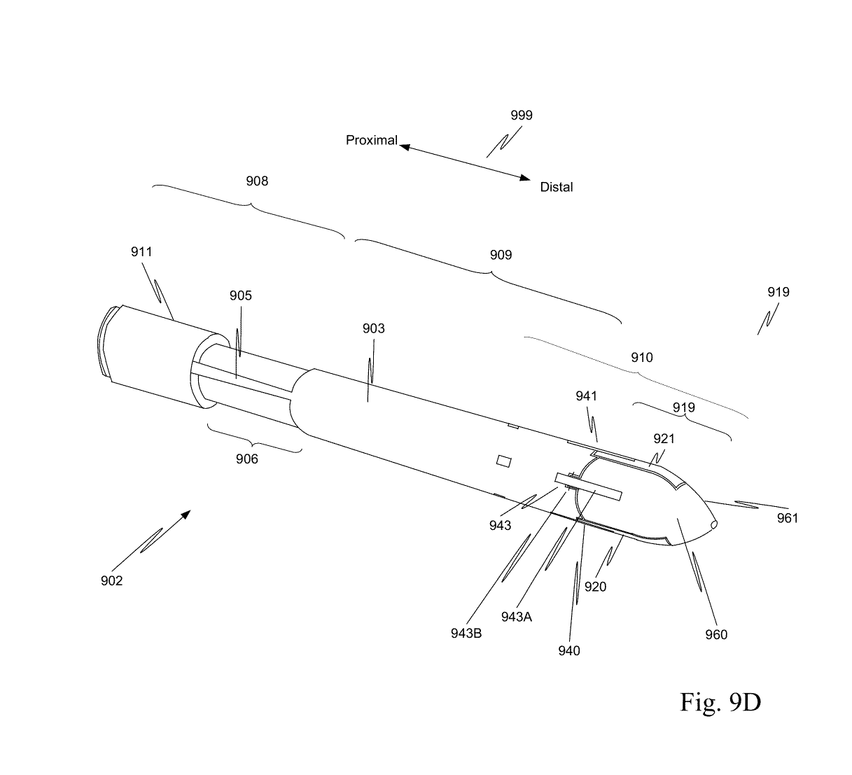

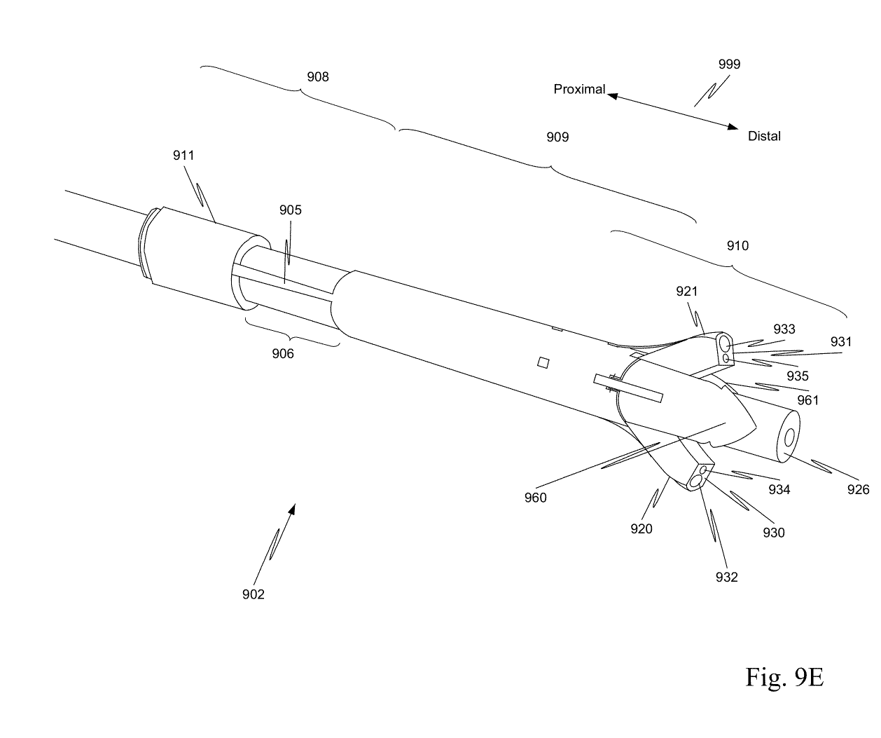

In FIGS. 9A to 9E, surgical instrument 202 is a cannula 902 with an obturating tip 919. In FIG. 9A, cannula 902 is transparent so that details of features inside cannula 902 are visible.

Cannula 902 includes a proximal portion 908, a body 909 extending distally from proximal portion 908, and a distal end portion 910 that includes a distal portion of body 909. As indicated by arrow 999, the distal direction is towards a surgical site and the proximal direction is away from the surgical site.

Proximal portion 908 of cannula 902 includes a power, control, and video interface (not shown), orientation marker 905, and engagement structure 906. The power, control, and video interface, orientation marker 905, and engagement structure 906 are equivalent to power, control, and video interface 204, orientation marker 205, and engagement structure 206, respectively, which were described above and so that description is incorporated herein by reference.

In this example, engagement structure 906 has a smaller outer diameter than the outer diameter of body 909. Cables from image capture sensors (not shown) are routed within a channel 914 in the cylindrical wall of distal end portion 910, body 909, and orientation marker 905 to collar 911. A different orientation marker or markers can be used on cannula 902, such as those described above.

In this example, body 909 has an outer circumferential surface 903 with an outer radius. Distal end portion 910 includes image capture elements 920, 921 and obturating tip 919. Obturating tip 919 is formed by two puncture tip elements 960, 961, in this aspect.

In this aspect, image capture elements 920, 921 are movably mounted on a distal end of body 909. Similarly, puncture tip elements 960, 961 are movably mounted on the distal end of body 909.

Each of image capture elements 920, 921 and puncture elements 960, 961 is connected to distal end portion 910 by a hinge element 940, 941, 942, and 943, respectively. In this example, hinge elements 940, 941, 942, 943 are implemented with a pivot pin and a flexure element. However, hinge elements 940, 941, 942, 943 can be implemented, for example, using any one of a joint, a living hinge with a spring element, a flexure, and a pivot pin and a flexure element.

Hinge element 943 is used as an example of one implementation of a hinge element. Hinge element 943 includes a pivot pin 943B and a flexure element 943A. Pivot pin 943B extends through a channel in a tab of puncture tip element 960. The tab of puncture element 960 fits in a slot in the distal end body 909. Pivot pin 943B is mounted in holes in the sides of the slot in body 909.

Flexure element 943A is a rectangular shaped metal strip, e.g., a stainless steel strip. Flexure element 943A sits in a groove formed in the outer surface of body 909 and in the outer surface of puncture tip element 960. Near the proximal end of the groove in outer surface 903, a portion of outer surface of body 909 extends over the grove to hold flexure element 943A in the groove.

In this aspect, each image capture element 920, 921 includes an image capture sensor and a lens. Each image capture element 920, 921 also includes an illumination device. In one aspect, the illumination device includes a light emitting diode on a semiconductor chip and a refracting element. The image capture sensor and the lens is a small camera. The cameras used in image capture elements 920, 921 are similar to those described above.

A first image capture sensor is positioned within image capture element 921 to capture light that passes through a first aperture 933 in edge surface 931 (FIG. 9B) of image capture element 921. Aperture 933 and the first image capture sensor are proximal to the distal tip of obturating tip 919. A second image capture sensor is positioned within image capture element 920 to capture light that passes through a first aperture 932 in edge surface 930 (FIGS. 9A and 9E) of image capture element 920. Aperture 930 and the second image capture sensor also are proximal to the distal tip of obturating tip 919.

A first illumination device is positioned within image capture element 920 so that light from the illumination device passes through a second aperture 934 (FIG. 9E) in edge surface 930 of image capture element 920. Similarly, a second illumination device is positioned within image capture element 921 so that light from the illumination device passes through a second aperture 935 (FIG. 9E) in edge surface 931 of image capture element 921.

In FIG. 9A, an insertion lock 916 is inserted in the central channel of cannula 902. Insertion lock 916 includes a locking mechanism that prevents puncture tip elements 960, 961 and image capture elements 920, 921 from moving radially outward, i.e., flexing outward, as cannula 902 is inserted into a patient. In this aspect, insertion lock 916 has a cylindrical body 916B extending distally from a disk 916A. At the distal end of cylindrical body 916B is a pointed tip 916T. FIG. 9B is a top view of pointed tip 916T. FIG. 9C is side view of pointed tip 916T.

Pointed tip 916T includes a plurality of post elements 916P1, 916P2, 916P3, and 916P4. Post element 916P1 has a tapered end that engages a hole in a proximal end of image capture element 920. Post element 916P2 has a tapered end that engages a hole in a proximal end of puncture tip element 960. Post element 916P3 has a tapered end that engages a hole in a proximal end of image capture element 921. Post element 916P4 has a tapered end that engages a hole in a proximal end of puncture tip element 961.

The use of a post and hole locking mechanism is illustrative only and is not intended to be limiting. Any locking mechanism can be used that prevents outward radial movement of image captures elements 920, 921 and puncture tip elements 9601, 961 during insertion of cannula 902. After cannula 902 is inserted in a patient, insertion lock 916 is withdrawn from cannula 902.

FIGS. 9A and 9D illustrates hinge elements 940, 941, 942, 943 in the first state with image capture elements 920, 921 un-deployed. After insertion lock 916 is removed from cannula 902, and when a device is not inserted through the central channel in cannula 902 (FIG. 9D), or is not inserted distally beyond hinge elements 940, 941, hinge elements 940, 941 automatically maintain image capture elements 920, 921 in the first state. Hinge elements 940, 941 automatically return image capture elements 920, 921 to the first state when a device is withdrawn from the central channel of cannula 902.

Thus, in the first state, the image capture sensors are enclosed within the outer circumferential surface of cannula 902. Thus, edge surfaces 930 and 931 having apertures 932 and 933, respectively, do not extend beyond the outer circumferential surface in the first state. The enclosure of the image capture sensors within the outer circumferential surface means that the overall size and length of cannula 902 is similar to prior art obturators that do not include image capture elements 920 and 921.

When device 926 is passed through a central channel in cannula 902, (FIG. 9E) device 926 displaces image capture elements 920, 921 so that apertures 932, 933 are moved to a position outside outer circumferential surface 903. Aperture 933 and aperture 935 in edge surface 931 are external to outer circumferential surface 903. Also, aperture 932 and aperture 934 in edge surface 930 are external to outer circumferential surface 903. Edge surfaces 930 and 931 form part of the distal end outer surface of cannula 902 in the second state.

Thus, image capture elements 920, 921 are deployed automatically by passing device 926 through cannula 902. Following deployment when the distal end of device 926 is moved proximal to hinge elements 940, 941, image capture elements 920, 921 automatically return to the first state without any action by a user of, or by the control system of the minimally invasive surgical system in which cannula 902 is mounted.

In FIGS. 11A and 11B, surgical instrument 202 is a cannula 1102. Cannula 1102 includes a proximal portion (not shown), and a body 1109 extending distally from the proximal portion. Body 1109 includes a distal end portion 1110.

The proximal portion of cannula 1102 includes a power, control, and video interface, an orientation marker, and an engagement structure. The power, control, and video interface, the orientation marker, and the engagement structure are equivalent to power, control, and video interface 204, orientation marker 205, and engagement structure 206, respectively, which were described above and so that description is incorporated herein by reference.

In this example, body 1109 has an outer circumferential surface 1103 with an outer radius R. Image capture elements 1120, 1121 are movably mounted on distal end portion 1110. Image capture elements 1120, 1121 are connected to distal end portion 1110 by hinge elements 1140, 1141, respectively. Hinge elements 1140, 1141 are implemented, for example, as a pivot pin and a spring element.

FIG. 11A illustrates hinge elements 1140, 1141 in the first state with image capture elements 1120, 1121 un-deployed. When there is not another device inserted through the central channel of cannula 1102, hinge elements 1140, 1141 automatically maintain image capture elements 1120, 1121 in the first state. Hinge elements 1140, 1141 automatically return image capture elements 1120, 1121 to the first state when a device is withdrawn from the central channel.

In this aspect, each image capture element 1120, 1121 includes an image capture sensor and a lens. The image capture sensor and the lens are a small camera.

The cameras used in image capture elements 1120, 1121 are similar to those available in cell phones and other small portable devices. The focal length of the camera is selected according to the desired field of view. The images captured by the camera typically have lower resolution than the images captured using an endoscope since the requirement on the resolution for peripheral vision is lower than the requirement on the resolution for fovea vision.

A second image capture sensor is positioned within image capture element 1121 to capture light that passes through a first aperture 1133 in edge surface 1131 of image capture element 1121. The second image capture sensor is proximal to distal edge surface 1131, which is part of the distal end surface of cannula 1102. In this aspect, the distal end surface is perpendicular to the longitudinal axis of cannula 1102. The longitudinal axis extends from the proximal end surface of cannula 1102 to the distal end surface of cannula 1102.

In the first state, the second image capture sensor is enclosed within outer radius R of cannula 1102 and within outer circumferential surface 1103 of body 1109. Thus, edge surface 1131 having aperture 1133 does not extend beyond outer circumferential surface 1103 in the first state.

Similarly, a first image capture sensor 1122 is positioned within image capture element 1120 to capture light that passes through a first aperture 1132 in edge surface 1130 of image capture element 1120. The first image capture sensor to distal edge surface 1130, which is part of the distal end surface of cannula 1102.

In the first state, the first image capture sensor is enclosed within the outer radius R of cannula 1102 and within outer circumferential surface 1103 of body 1109. Thus, edge surface 1130 having aperture 1132 does not extend beyond outer circumferential surface 1103 in the first state. The enclosure of edge surface 1131, the second image capture sensor, edge surface 1130, and the first image capture sensor within outer circumferential surface 1103 means that the outer diameter and length of cannula 1102 are similar to prior art cannulas that do not include image capture elements 1120 and 1121.

In this aspect, edge surface 1130 of image capture element 1120 and edge surface 1131 of image capture element 1121 have a yin-yang like shape. Each edge surface has two equal oppositely oriented semicircles of radius R/2 joined at their edges (Point C), plus a semicircle of radius R joining the other outer edges of the two semicircles. Note that the edge surfaces are not exactly a yin-yang shape due to the space required for the hinge element and so edge surfaces 1130, 1131 are referred as having yin-yang like shapes. In FIG. 11B, this shape extends longitudinally along image capture elements 1120, 1121 between edge surfaces 1130, 1131 and proximal edge surfaces of image capture elements 1120, 1121. This is illustrative only and is not intended to limiting. The yin-yang like shape could extend only part of the way between edge surface 1130, 1131 and the proximal edge surfaces of image capture elements 1120, 1121, for example.

When device 1126 is passed through the central channel of cannula 1102, (FIG. 11B) device 1126 displaces image capture elements 1120, 1121 so that the image capture sensors are moved to a position outside an inner radius of cannula 1102. Aperture 1133 in edge surface 1131 is external to outer circumferential surface 1103, i.e., any point on the border of aperture 1133 is a distance greater than the outer radius R from the longitudinal axis. Also, aperture 1132 in edge surface 1130 is external to outer circumferential surface 1103, i.e., any point on the border of aperture 1132 is a distance greater than outer radius R from the longitudinal axis.

Thus, image capture elements 1120, 1121 are deployed automatically by passing device 1126 through cannula 1102. Following deployment when the distal end of device 1126 is moved proximal to hinge elements 1140, 1141, image capture elements 1120, 1121 automatically return to the first state without any action by a user of the minimally invasive surgical system, or by the control system of the minimally invasive surgical system in which cannula 1102 is mounted.

In the above examples, the image capture elements were automatically deployed by insertion of a device through the central channel of the cannula. In some aspects, a mechanical mechanism is used to deploy the image capture elements. For example, cables or linkage are attached to each of the image capture elements. Manipulation of the cables or linkage is used to deploy the image capture elements. A twist mechanism could be included in the proximal portion of the cannula. The twist mechanism is connected by the cables or linkage to each of the image capture elements. When the twist mechanism is rotated in a first direction around a central axis of the cannula, the image capture elements are deployed. When the twist mechanism is rotated in a second direction that is opposite to the first direction around the central axis of the cannula, the image capture elements are retracted. Other mechanisms could be coupled to the cables or linkage attached to the image capture elements to deploy and retract the image capture elements.

All examples and illustrative references are non-limiting and should not be used to limit the claims to specific implementations and embodiments described herein and their equivalents. Any headings are solely for formatting and should not be used to limit the subject matter in any way, because text under one heading may cross reference or apply to text under one or more headings. Finally, in view of this disclosure, particular features described in relation to one aspect or embodiment may be applied to other disclosed aspects or embodiments of the invention, even though not specifically shown in the drawings or described in the text.

The various modules described herein can be implemented by software executing on a processor, hardware, firmware, or any combination of the three. When the modules are implemented as software executing on a processor, the software is stored in a memory as computer readable instructions and the computer readable instructions are executed on the processor. All or part of the memory can be in a different physical location than a processor so long as the processor can be coupled to the memory. Memory refers to a volatile memory, a non-volatile memory, or any combination of the two.

Also, the functions of the various modules, as described herein, may be performed by one unit, or divided up among different components, each of which may be implemented in turn by any combination of hardware, software that is executed on a processor, and firmware. When divided up among different components, the components may be centralized in one location or distributed across system 300 for distributed processing purposes. The execution of the various modules results in methods that perform the processes described above for the various modules.

Thus, a processor is coupled to a memory containing instructions executed by the processor. This could be accomplished within a computer system, or alternatively via a connection to another computer via modems and analog lines, or digital interfaces and a digital carrier line.

Herein, a computer program product comprises a computer readable medium configured to store computer readable code needed for any part of or all of the processes described herein, or in which computer readable code for any part of or all of those processes is stored. Some examples of computer program products are CD-ROM discs, DVD discs, flash memory, ROM cards, floppy discs, magnetic tapes, computer hard drives, servers on a network and signals transmitted over a network representing computer readable program code. A non-transitory tangible computer program product comprises a tangible computer readable medium configured to store computer readable instructions for any part of or all of the processes or in which computer readable instructions for any part of or all of the processes is stored. Non-transitory tangible computer program products are CD-ROM discs, DVD discs, flash memory, ROM cards, floppy discs, magnetic tapes, computer hard drives and other physical storage mediums.

In view of this disclosure, instructions used in any part of or all of the processes described herein can be implemented in a wide variety of computer system configurations using an operating system and computer programming language of interest to the user.

Herein, first and second are used as adjectives to distinguish between elements and are not intended to indicate a number of elements. Also, top, bottom, and side are used as adjectives to aid in distinguishing between elements as viewed in the drawings, and to help visualize relative relationships between the elements. For example, top and bottom surfaces are first and second surfaces that are opposite and removed from each other. A side surface is a third surface that extends between the first and second surfaces. Top, bottom, and side are not being used to define absolute physical positions.

The above description and the accompanying drawings that illustrate aspects and embodiments of the present inventions should not be taken as limiting—the claims define the protected inventions. Various mechanical, compositional, structural, electrical, and operational changes may be made without departing from the spirit and scope of this description and the claims. In some instances, well-known circuits, structures, and techniques have not been shown or described in detail to avoid obscuring the invention.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of the device in use or operation in addition to the position and orientation shown in the figures. For example, if the device in the figures is turned over, elements described as “below” or “beneath” other elements or features would then be “above” or “over” the other elements or features. Thus, the exemplary term “below” can encompass both positions and orientations of above and below. The device may be otherwise oriented (rotated 90 degrees or at other orientations) and the spatially relative descriptors used herein interpreted accordingly. Likewise, descriptions of movement along and around various axes include various special device positions and orientations.

The singular forms “a”, “an”, and “the” are intended to include the plural forms as well, unless the context indicates otherwise. The terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

We claim:

1. An apparatus comprising:
a surgical instrument comprising:
a distal end portion having an outer surface with an outer radius;
one or more image capture elements movably mounted in the distal end portion, each image capture element comprising: a surface having an aperture; and an image capture sensor positioned within the image capture element to receive light that passes through the aperture in the surface,
wherein in a first state, the one or more image capture elements is un-deployed, and the surface having the aperture of at least one of the one or more image capture elements is enclosed within the outer surface so that the surface having the aperture does not extend beyond the outer surface, and
wherein in a second state, the one or more image capture elements is deployed, and the surface having the aperture of the at least one of the one or more image capture elements is positioned beyond the outer radius and so extends beyond the outer surface.

2. The apparatus of claim 1, at least one image capture element further comprising an illuminator.

3. The apparatus of claim 1, the surgical instrument further comprising:
an orientation marker fixed in position relative to the one or more image capture elements.

4. The apparatus of claim 1, the surgical instrument further comprising:
a proximal end portion; and
a clamp engagement structure positioned on the proximal end portion.

5. The apparatus of claim 1, further comprising:
a portable display unit attached to a proximal end portion of the surgical instrument.

6. The apparatus of claim 1, the surgical instrument further comprising:
a hinge element connecting one image capture element in the one or more image capture elements to the distal end portion.

7. The apparatus of claim 6, the surgical instrument comprising a cannula including a obturating tip.

8. The apparatus of claim 7, the obturating tip further comprising:
a plurality of distal end puncture tip elements, each of the puncture tip elements movably connected to the distal end portion.

9. The apparatus of claim 6, the surgical instrument comprising a cannula.

10. The apparatus of claim 9, the surgical instrument further comprising a sheath extending through the cannula, wherein the sheath includes the one or more image capture elements.

11. The apparatus of claim 6, further comprising:
an elongate rod including an illuminator, wherein upon passing the elongate rod through the surgical instrument, the one or more of image capture elements move from the first state to the second state.

12. The apparatus of claim 11, further comprising:
a controller coupled to each image capture element in the one or more image capture elements, wherein the controller generates a panoramic image from images captured by the one or more image capture elements.

13. The apparatus of claim 12, further comprising:
a portable display unit attached to the elongate rod proximal to the surgical instrument.

14. The apparatus of claim 6, further comprising:
an endoscope, wherein upon passing the endoscope through the surgical instrument, the one or more image capture elements move from the first state to the second state.

15. The apparatus of claim 14, further comprising:
a controller coupled to each image capture element in the one or more image capture elements and to an image capture unit coupled to the endoscope, wherein the controller generates a panoramic image from images captured by the one or more image capture elements and sends an image captured by the image capture unit coupled to the endoscope and the panoramic image to a display device.

16. The apparatus of claim 15, wherein the panoramic image includes a footprint of the image captured from the endoscope.

17. A method comprising:
positioning a surgical instrument,
wherein the surgical instrument includes one or more image capture elements in an un-deployed state, and
wherein in the un-deployed state, an image capture sensor in at least one of the one or more image captures elements is proximal to a distal end surface of the surgical instrument and enclosed within an outer diameter of the instrument;
inserting a device through the surgical instrument to deploy the one or more image capture elements; and
generating a panoramic image from images captured by the one or more deployed image capture elements.

18. The method of claim 17, further comprising:
placing a port in a patient using the panoramic image.

19. The method of claim 17, wherein the inserting a device comprises:
inserting an elongate rod including an illuminator through the surgical instrument so that a distal end of the elongate rod extends beyond a distal end of the surgical instrument.

20. The method of claim 17, further comprising:
engaging the surgical instrument to a manipulator arm of a minimally invasive surgical system to orient the one or more image capture elements in a known orientation.

21. The method of claim 17, the inserting a device comprises:
inserting an endoscope through the surgical instrument so that a distal end of the endoscope extends beyond a distal end of the surgical instrument.

22. The method of claim 21, further comprising:
combining an image captured from the endoscope with the panoramic image with an orientation of the panoramic image rotated to the orientation of the image captured from the endoscope in the combination image; and
displaying the combination image to an operator of the endoscope.

23. The method of claim 21, further comprising:
combining an image captured from the endoscope with the panoramic image with an orientation of the image captured from the endoscope rotated to the orientation of the panoramic image in the combination image; and
displaying the combination image on a display different from a display used by an operator of the endoscope.

24. The method of claim 21, further comprising:
blending an image captured from the endoscope with the panoramic image.

25. The method of claim 21, further comprising:
indicating a footprint of an image captured from the endoscope in the panoramic image.

26. The method of claim 17, the surgical instrument comprising a cannula.

27. The method of claim 17, the surgical instrument comprising a cannula having an obturating tip.

28. The method of claim 17, the surgical instrument comprising a sheath extending through a cannula.

29. The method of claim 17, wherein image capture elements in the one or more image capture elements are positioned at different distances from a proximal end of the surgical instrument.
